(19) United States
(12) Patent Application Publication
(10) Pub. No.: US 2020/0113899 A1
(43) Pub. Date: Apr. 16, 2020

(54) STATIN COMPOSITIONS AND METHODS FOR USE IN TREATING SYNUCLEINOPATHIES

(71) Applicant: CHASE THERAPEUTICS CORPORATION, Washington, DC (US)

(72) Inventors: Thomas N. CHASE, Washington, DC (US); Kathleen E. CLARENCE-SMITH, Washington, DC (US)

(73) Assignee: CHASE THERAPEUTICS CORPORATION, Washington, DC (US)

(21) Appl. No.: 16/613,013

(22) PCT Filed: Jul. 3, 2018
(86) PCT No.: PCT/US2018/040665

§ 371 Date: Nov. 12, 2019

Related U.S. Application Data
(60) Provisional application No. 62/528,204, filed on Jul. 3, 2017.

(51) Int. Cl. A61K 31/505; A61K 31/428; A61K 31/404; A61K 31/37; A61K 31/47; A61K 31/22; A61P 25/16; A61P 25/28

(52) U.S. Cl. CPC A61K 31/505; A61K 31/428; A61K 31/404; A61P 25/28; A61K 31/47; A61K 31/22; A61P 25/16; A61K 31/37

(57) ABSTRACT

The present invention describes pharmaceutical combinations, compositions, and methods comprising a statin and 6-propylamino-4,5,6,7-tetrahydro-1,3-benzothiazole-2-amine or a pharmaceutically acceptable salt and/or solvate thereof, that are useful for the treatment of synucleinopathic disorders.

### RELATED APPLICATIONS

This application claims the benefit of U.S. Provisional Patent Application Ser. No. 62/528,204, filed Jul. 3, 2017, the disclosure of which is incorporated herein by reference in its entirety.

### FIELD OF THE INVENTION

This invention pertains to the field of the treatment of synucleinopathies, i.e. of neurodegenerative disorders of the human central nervous system, and in particular of the treatment of neurotoxic processes due to alpha-synuclein oligomerization and aggregation.

### OBJECT OF THE INVENTION

The present invention concerns a pharmaceutical combination comprising a statin and 6-propylamino-4,5,6,7-tetrahydro-1,3-benzothiazole-2-amine or a pharmaceutically acceptable salt or solvate thereof, including fixed-dose combinations, and its use for the treatment of synucleinopathies, in particular of the CNS neurotoxic effects of alpha-synuclein in humans. A preferred embodiment of the present invention includes the use of a statin for augmenting the synucleinopathy-modifying potential of pramipexole in humans, thus allowing at least a slowing of disease progression at doses that are both safe and tolerable.

### Definitions

“CNS”: Central Nervous System.
“IR”: Immediate Release of the active ingredient from a composition.
“ER”: Extended Release of the active ingredient from a composition.
“GI”: Gastro-Intestinal.
“AE(s)”: Adverse Effect(s).
“SNCA”: Synuclein-alpha or alpha-synuclein.
“MSA”: Multiple System Atrophy.
“PD”: Parkinson's Disease.
“LBD”: Lewy Body Dementia.
“TTS”: Transdermal Therapeutic System.
“Synucleinopathy”: a disease characterized by the abnormal accumulation, processing, and spreading of alpha-synuclein (α-synuclein) in the brain. Synucleinopathies (also called α-synucleinopathies) are neurodegenerative diseases which include, but are not limited to Parkinson's disease, Lewy body dementia (LBD), dementia with Lewy bodies (DLB), Alzheimer's disease (AD), the Lewy body variant of AD, multiple system atrophy, neurodegeneration with brain iron accumulation, and parkinsonian disorders associated with glucocerebrosidase (GBA) mutations.
“Dyslipidemia”: a disorder of lipoprotein metabolism, including lipoprotein overproduction or deficiency, as may be manifested by elevation of the total cholesterol, the low-density lipoprotein (LDL) cholesterol and the triglyceride concentrations, and a decrease in the high-density lipoprotein (HDL) cholesterol concentration in the blood, and other blood disorders the statins are indicated for.
“Pramipexole”: a general term that, unless otherwise specified, designates the (S)-6-propylamino-4,5,6,7-tetrahydro-1,3-benzothiazole-2-amine active principle per se, and includes the free base and the salts and solvates thereof.
“Statin”: a class of chemical compounds with a 3,5-dihydroxyheptane or 3,5-dihydroxyhept-6-ene carboxylic acid structure linked, via its 7-position, to a carbocyclic or heterocyclic structure, in some cases in form of 5-lactone thereof, used as medicaments for treating dyslipidemia.
“Effective statin dose/per unit form (or dose per unit form)” and “Effective statin daily dose”: a statin dose per unit form or daily dose of from 0.5 mg to 80 mg. According to the structure of each statin, said dose-range refers to an equivalent of the free acid, to an equivalent of a specific salt, or, in case of a lactone, to the lactone itself.
“Effective pramipexole dose/unit form”: a dose per unit form of pramipexole or pharmaceutically acceptable salt thereof that is equivalent to at least from 0.125 mg to 6 mg of pramipexole dihydrochloride monohydrate.
“Effective pramipexole daily dose”: a daily dose (in pramipexole dihydrochloride monohydrate) including doses used during titration period, that is at least as high as a daily dose approved for the symptomatic treatment of PD (from 0.375 mg/day to 4.5 mg/day of pramipexole dihydrochloride monohydrate).
“6-Propylamino-4,5,6,7-tetrahydro-1,3-benzothiazole-2-amine”: A chiral chemical compound that is available as racemate, chemically (R,S)-6-propylamino-4,5,6,7-tetrahydro-1,3-benzothiazole-2-amine, as (R)-stereoisomer, chemically (R)-6-propylamino-4,5,6,7-tetrahydro-1,3-benzothiazole-2-amine (“dexpramipexole”, INN), and as (S)-stereoisomer, chemically (S)-6-propylamino-4,5,6,7-tetrahydro-1,3-benzothiazole-2-amine (“pramipexole”, INN). These three chemical entities are basic substances that may be isolated each as an acid addition salt and solvate thereof. Pramipexole dihydrochloride monohydrate is also known with its USAN “pramipexole hydrochloride”. As used herein, “6-propylamino-4,5,6,7-tetrahydro-1,3-benzothiazole-2-amine” is a general term that, unless otherwise specified, designates a member selected from the group consisting of pramipexole, dexpramipexole, the racemate, and pramipexole/dexpramipexole mixtures ((S)/(R)-mixtures)) or combinations ((R)-(S) combinations). The 6-propylamino-4,5,6,7-tetrahydro-1,3-benzothiazole-2-amine daily and per unit form dose are expressed as equivalents of pramipexole dihydrochloride monohydrate also when referred to as dexpramipexole, (R)-(S) combinations and (S)/(R)-mixtures.
“(R)/(S)-mixture”: This term designates a dexpramipexole/pramipexole physical mixture used as an active ingredient according to the present invention.
“(S)-enantiomer”: this term, as used herein with reference to 6-propylamino-4,5,6,7-tetrahydro-1,3-benzothiazole-2-amine doses (daily or per unit form) designates the (S)-stereoisomer, included in said doses that, in said 6-propylamino-4,5,6,7-tetrahydro-1,3-benzothiazole-2-amine, are primarily responsible for its dopaminergic action. More specifically, “S-enantiomer” is herein used to designate the S-stereoisomer that is present in the racemate or pharmaceutically acceptable salt thereof, and similarly, to designate the pramipexole or pharmaceutically acceptable salt thereof that is present, as (S)-constituent, in a (S)/(R)-mixture or in (S)-(R) combination, in order to distinguish it from pramipexole used alone.
“Effective 6-propylamino-4,5,6,7-tetrahydro-1,3-benzothiazole-2-amine dose/unit form”: a given dose, or dose-range, per unit form of 6-propylamino-4,5,6,7-tetrahydro-1,3-benzothiazole-2-amine, as defined above, equivalent to from 0.125 mg to 3000 mg of pramipexole dihydrochloride monohydrate, wherein pramipexole, as such or as (S)-enantiomer, is present in an amount equivalent to from 0.125 mg to 20 mg of pramipexole dihydrochloride monohydrate.
“Effective 6-propylamino-4,5,6,7-tetrahydro-1,3-benzothiazole-2-amine daily dose”: a daily dose of 6-propylamino-4,5,6,7-tetrahydro-1,3-benzothiazole-2-amine, as defined above, of from 0.375 mg to 3000 mg, including an effective pramipexole, as such or as (S)-enantiomer, daily dose as defined above.

The term “comprise,” “comprises,” “comprising” “include,” “includes,” and “including” are interchangeable and not intended to be limiting.

It is to be further understood that where descriptions of various embodiments use the term “comprising,” those skilled in the art would understand that the present disclosure also contemplates such embodiments alternatively described using the language “consisting essentially of” or “consisting of”.

### BACKGROUND OF THE INVENTION

Alpha-synuclein, a protein composed of 140 amino acids encoded by the SNCA (Synuclein-Alpha) gene, is abundantly expressed in the human brain and to a lesser extent in various other organs. In brain, alpha-synuclein (hereafter also referred to as “synuclein”) is mainly found in neuronal terminals, especially in the cortex, hippocampus, substantia nigra and cerebellum, where it contributes to the regulation of neurotransmitter release, and passes into the peripheral blood stream (Marques and Outeiro, 2012), in part packaged with exosomal vesicles originating from the CNS (Shi et al, 2014).

Under normal circumstances, this soluble protein forms a stably folded tetramer that resists aggregation. But, in certain pathological conditions, for unknown reasons, the alpha-synuclein oligomerizes and aggregates (with the formation of fibrils or “fibrillization”). Somewhere along this aberrant pathway, toxic synuclein species are believed to be formed which also pass into the peripheral (systemic) circulation, carried within exosomes.

Aberrant alpha-synuclein oligomerization and aggregation are thought to be the cause of synucleinopathies, notably Parkinson's disease, Lewy body dementia, Dementia with Lewy Bodies (DLB), parkinsonian disorders associated with glucocerebrosidase (GBA) mutations, multiple system atrophy (MSA), some forms of Alzheimer's disease (AD), and several other disorders, which are collectively referred to as “synucleinopathies”. Alpha-synuclein is a ubiquitous protein that is especially abundant in the brain and has been postulated to play a central role in the pathogenesis of Parkinson's disease (PD), Alzheimer's disease, and other neurodegenerative disorders (Kim et al. 2004).

Several other disorders have also, albeit less frequently, been considered synucleinopathies. These include Hallevorden-Spatz syndrome, neuronal axonal dystrophy and some cases of traumatic brain injury. In the case of Hallevorden-Spatz syndrome, symptoms include parkinsonism, dystonia, dysphagia/dysarthria, rigidity/stiffness of limbs, dementia, and spasticity.

Many now believe that processes leading to synuclein aggregation may be central to the neuronal injury and destruction occurring in these disorders.

Statins, such as atorvastatin, available in 10 mg, 20 mg, 40 mg and 80 mg tablets and administered at a daily dose from 10 mg to 80 mg; fluvastatin, available in capsules containing fluvastatin sodium, equivalent to 20 mg, 40 mg or 80 mg of fluvastatin, for oral administration, or ER-tablets containing fluvastatin sodium, equivalent to 80 mg of fluvastatin, and administered at a daily dose of 20 mg-80 mg; lovastatin available in 20 mg and 40 mg tablets, and administered at the recommended dosing range of 10-80 mg/day; pitavastatin available in 1 mg, 2 mg and 4 mg tablets, and administered once a day at a daily dose range of from 1 mg to 4 mg; pravastatin available in 10 mg, 20 mg, 40 mg, and 80 mg tablets, and administered at a daily dose from 10 mg to 80 mg; simvastatin available in 5 mg, 10 mg, 20 mg, 40 mg and 80 mg tablets, and administered at a daily dose of from 5 mg to 80 mg, normally from 5 mg to 40 mg; and rosuvastatin, available in 5 mg, 10 mg, 20 mg, and 40 mg tablets, and administered at a daily dose-range of from 5 mg to 40 mg, are indicated for dyslipidemia, and in particular for the reduction of blood total cholesterol, blood LDL cholesterol and triglyceride levels as well as to raise HDL cholesterol levels.

For nearly three decades, statins have been regarded as safe and effective in the primary and secondary prevention of cardiovascular disease, especially for reducing the risk of heart attacks, stroke, and certain arterial revascularization procedures.

Studies in cultured human cells as well as in animal models have shown drugs of this class are copiously yet selectively taken up by the liver, the target organ for cholesterol lowering drugs. Within the liver, it is currently believed that the lipid-modifying effects of statins such as rosuvastatin occur as a result of increasing the number of hepatic LDL receptors on cell-surfaces to enhance the uptake and catabolism of LDL as well as by inhibiting the hepatic synthesis of very low-density lipoproteins (VLDL).

Statins act selectively as competitive inhibitors of HMG-CoA reductase, the rate-limiting enzyme that converts 3-hydroxy3methylglutaryl coenzyme A to mevalonate, a precursor in the synthesis of cholesterol (see for example the Prescribing Information, Crestor [rosuvastatin calcium] tablets. Revised: 20 May 2016).

Pharmaceutical agents currently proposed for consideration as possible candidates for the treatment of synucleinopathies include pramipexole and its analogues, alone or in combination with various drugs. Pramipexole is a synthetic aminothiazole derivative described in U.S. Pat. No. 4,886,812, the contents of which are incorporated herein in their entirety by reference. It is a dopamine autoreceptor agonist (Schneider C S and Mierau J, 1987) that has been approved since the late 1990s for the symptomatic treatment of Parkinson's disease (PD) in doses ranging from 0.375 mg/day to 4.5 mg/day, given in 3 equally divided doses (Mirapex Prescribing Information, July 2016.

More recently, it began to be reported that pramipexole can exert neuroprotective effects in various in vitro cellular and in vivo animal models of PD and also in a Phase I study (US 2013/0116292, see below). Mechanisms by which these protective effects may occur remain uncertain. Unfortunately, the protective effects of pramipexole in animal models are generally small and require higher doses than considered safe and tolerable for human administration. It thus hardly surprising that pramipexole failed to evidence neuroprotective (i.e., disease modifying) activity in a randomized, controlled, clinical trial involving 535 PD patients (Schapira A H 2013).

Pramipexole treatment has also been reported to modify the concentration of alpha-synuclein (hereafter termed “synuclein”) species contained within exosomes collected from the peripheral blood of PD patients (Bar-On et al. 2008, Luo H T et al. 2016), changes considered indicative of the characteristic pathologic alterations occurring in the brain of those suffering from this disorder (Shi et al, 2014). Nevertheless, these synuclein biomarker changes appeared relatively modest in magnitude and occurred only in those titrated to the maximum recommended dose of pramipexole. The foregoing observations lend further support to the view that pramipexole monotherapy is not a safe and effective approach to the neuroprotective therapy of patients with synucleinopathic disorders of the PD type.

The neuroprotective activity of (R)-propylamino-4,5,6,7-tetrahydro-1,3-benzothiazole-2-amine or pharmaceutically acceptable salts and solvates thereof, that is not significantly dopaminergic, is disclosed in US 2013/0116292, the contents of which are incorporated herein in their entirety by reference. According to this document, said (R)-propylamino-4,5,6,7-tetrahydro-1,3-benzothiazole-2-amine, or pharmaceutically acceptable salts and solvates thereof, acts by slowing the progression of neuronal degeneration and/or by preventing neuronal cell death. However, no further mention of this possible action of the (R)-isomer of pramipexole appeared in the literature.

A synthesis of (R)-propylamino-4,5,6,7-tetrahydro-1,3-benzothiazole-2-amine (also called dexpramipexole) and of pharmaceutically acceptable salts thereof, in particular dexpramipexole dihydrochloride monohydrate, is described in US 2012/0253047, the contents of which are incorporated herein in their entirety by reference.

(S)-(R)-combinations and (S)/(R)-mixtures consisting of pharmaceutical compositions comprising a therapeutically effective amount of (R)-propylamino-4,5,6,7-tetrahydro-1,3-benzothiazole-2-amine or pharmaceutically acceptable salts and solvates thereof and a therapeutically effective amount of (S)-propylamino-4,5,6,7-tetrahydro-1,3-benzothiazole-2-amine or pharmaceutically acceptable salts and solvates thereof, useful for the treatment of PD, are disclosed in US 2008/00142590014259 (U.S. Pat. No. 8,017,598), the contents of which are incorporated herein in their entirety by reference. In particular, U.S. Pat. No. 8,017,598 discloses a method of treating and delaying the progression of Parkinson's disease or the symptoms thereof comprising administering to a subject in need thereof 100 milligrams to about 3,000 milligrams of R(+) pramipexole in combination with about 0.125 mg to about 1.5 milligrams of S(−) pramipexole. According to US 2008/0014259, both enantiomers are able to confer neuroprotective effects by their ability to accumulate in brain cells, the spinal cord and mitochondria where they exert a positive effect on neurological function that is independent of the dopamine agonist activity of pramipexole. Said document proposes said composition as a neuroprotective agent and a therapeutically effective amount of from about 0.0625 mg to about 6 mg of pramipexole in combination with up to 5000 mg of dexpramipexole. However, this document emphasizes the pramipexole adverse effects due to its dopaminergic action and tends to privilege pramipexole low doses, as also confirmed by the same applicant in the almost concurrent WO 2008/113003 document, the contents of which are incorporated herein in their entirety by reference. Also in this case, no further mention of this use of pramipexole (S)/(R) isomer combinations or mixtures appeared in the literature.

Unfortunately, limitations associated with the administration of pramipexole to synucleinopathic patients complicate its use at the potentially higher neuroprotective doses predicted by some animal models. First, mechanisms to explain its putatively beneficial effects on synuclein-related neurotoxicity continue to elude full understanding. Second, effect sizes in animal model studies tend to be small and occur only at relatively high drug doses. Both situations were also observed in the above mentioned report of pramipexole-induced changes in exosomal synuclein in PD patients, which were associated with the administration of the highest—4.5 mg/day—recommended/approved dose of pramipexole.

In the aforementioned report by Luo et al. (2016), although treatment of Parkinson patients with pramipexole at therapeutic doses significantly lowered the relative expression of alpha-synuclein (compared with pre-treatment values), the magnitude of the effect was small. Higher doses of pramipexole could have been more efficacious, but side effects such as vomiting and severe nausea preclude the use of higher doses. For example, Corrigan et al (2000) report that doses of 5 mg/day of pramipexole, hardly higher than the maximum recommended dose of 4.5 mg/day caused nausea in 76% of patients and vomiting in 39% of patients. Furthermore, 36% of patients were not able to complete the study, presumably because of intolerable GI adverse events.

Thus, the problem of providing safe, chronic, effective treatment of a patient suffering from a synucleinopathy with pramipexole has not yet been solved, since the current known treatment regimes for such patients do not significantly slow the progression of their fatal disorder.

### SUMMARY OF THE INVENTION

The present inventors have discovered that the effects of (S)-6-propylamino-4,5,6,7-tetrahydro-1,3-benzothiazole-2-amine (pramipexole) on the synuclein exosomal biomarker in the peripheral blood of patients with synucleinopathic disorders like PD are substantially and unexpectedly augmented by the co-administration of a statin. Not only does the effect size become clinically significant but the dose requirement for both drugs now falls into the range considered safe and tolerable for human subjects. In the present invention, the combination of pramipexole plus a statin safely interdicts the basic degenerative disease process in such patients to a clinically meaningful degree.

The forgoing observations are especially surprising since
statins, such as atorvastatin, fluvastatin, lovastatin, pitavastatin, pravastatin, simvastatin and rosuvastatin, were approved beginning in the late 1980s as lipid lowering agents;
the neuroprotective activity in PD models (Orr J D) and the reduction of neuronal alpha-synuclein aggregation (Bar-On et al.) was known from 2008;
no one suspected that a combination of a statin and 6-propylamino-4,5,6,7-tetrahydro-1,3-benzothiazole-2-amine, including pramipexole, could have been disease-modifying when administered to a patient suffering of a synucleinopathy;
no statin has been documented to confer disease modifying benefit to those with PD or a similar synucleinopathic disorder or to affect any peripheral exosomal biomarker of CNS disorders of this type;
6-propylamino-4,5,6,7-tetrahydro-1,3-benzothiazole-2-amine, such as pramipexole, has no known effects on the mevalonate pathway believed to be central to the lipid-lowering effects of the statins; indeed, statins and 6-propylamino-4,5,6,7-tetrahydro-1,3-benzothiazole-2-amine such as pramipexole are taken up, metabolized and excreted by different, essentially non-interactive, ways;
the HMG-CoA reductase inhibitor, as a statin, and the dopamine agonist, as pramipexole, act by different means on different body systems to produce different clinical effects in human subjects;
neither rosuvastatin nor any other drug of the HMG-CoA reductase inhibitor class is known to exert a synergistic effect on any pharmacologic action of pramipexole or of its isomer or of mixtures thereof, and
no one has suggested that these two drugs be co-administered to PD-type patients with neuroprotective intent.

It has also been found that, with the co-administration of a statin, not only did the pramipexole effect size become clinically significant but the dose requirement for either drug now fell into the range considered safe and tolerable for human subjects. These observations indicate that the combination of pramipexole, its stereoisomer, and mixtures thereof, plus a statin, safely interdict the basic degenerative disease process in synucleinopathic disorders to a clinically meaningful degree.

The combination of pramipexole plus a statin serves as the first neuroprotective treatment for those suffering from a parkinsonian synucleinopathic disorder, a goal long sought but never heretofore achieved.

Thus, the present invention provides a method for treating a patient suffering from a synucleinopathy, which comprises administering to a patient in need of said treatment an effective daily dose of a statin, in combination with an effective daily dose of 6-propylamino-4,5,6,7-tetrahydro-1,3-benzothiazole-2-amine.

The present invention also provides a statin, for use for combating synucleinopathies in a patient, in combination with pramipexole or a pharmaceutically acceptable salt thereof.

The invention further provides the use of a statin for the preparation of a medicament for combating synucleinopathies in a patient, in combination with 6-propylamino-4,5,6,7-tetrahydro-1,3-benzothiazole-2-amine, in particular pramipexole or a pharmaceutically acceptable salt thereof.

In addition, the invention provides the use of a statin for the preparation of a medicament for combating synucleinopathies in a patient, in a fixed-dose combination with 6-propylamino-4,5,6,7-tetrahydro-1,3-benzothiazole-2-amine, in particular with pramipexole or a pharmaceutically acceptable salt or solvate thereof.

In the treatment of a patient suffering from a synucleinopathy with a statin in combination with 6-propylamino-4,5,6,7-tetrahydro-1,3-benzothiazole-2-amine, said statin is administered at a daily dose of from 0.5 mg to 80 mg, and said 6-propylamino-4,5,6,7-tetrahydro-1,3-benzothiazole-2-amine is administered at a daily dose of from 0.375 mg to 3000 mg, including a daily dose of (S)-enantiomer equivalent to from 0.375 mg to 20 mg, normally from 0.375 mg to 6 mg of pramipexole dihydrochloride monohydrate.

In an embodiment, for this method (or use), said statin and said 6-propylamino-4,5,6,7-tetrahydro-1,3-benzothiazole-2-amine are each formulated in a pharmaceutical composition in admixture with a pharmaceutical carrier or vehicle.

In another embodiment, for the same method (or use), said statin and said 6-propylamino-4,5,6,7-tetrahydro-1,3-benzothiazole-2-amine are formulated in a pharmaceutical composition wherein said statin and said 6-propylamino-4,5,6,7-tetrahydro-1,3-benzothiazole-2-amine are mixed together and in admixture with a pharmaceutical carrier or vehicle.

In these compositions, said statin is present in a dose per unit form from half the aforementioned minimum dose per unit form to the maximum aforementioned dose per unit form approved for the treatment of dyslipidemia, normally in a dose/unit form of from 0.5 mg to 80 mg, normally from 2.5 mg to 80 mg, and said 6-propylamino-4,5,6,7-tetrahydro-1,3-benzothiazole-2-amine is present in an effective 6-propylamino-4,5,6,7-tetrahydro-1,3-benzothiazole-2-amine dose/unit form as defined above. Normally, said 6-propylamino-4,5,6,7-tetrahydro-1,3-benzothiazole-2-amine dose/unit form is equivalent to from 0.125 mg to 3000 mg of pramipexole dihydrochloride monohydrate, and includes an effective (S)-enantiomer dose/unit form as defined above (equivalent to from 0.125 mg to 20 mg, normally 0.125 mg to 6 mg, of pramipexole dihydrochloride monohydrate). If, in said 6-propylamino-4,5,6,7-tetrahydro-1,3-benzothiazole-2-amine dose/unit form, said (S)-enantiomer is present in a racemic 6-propylamino-4,5,6,7-tetrahydro-1,3-benzothiazole-2-amine, said 6-propylamino-4,5,6,7-tetrahydro-1,3-benzothiazole-2-amine dose/unit form is equivalent to from 0.25 mg to 3000 mg of pramipexole dihydrochloride monohydrate and includes an amount of racemate that is equivalent to from 0.25 mg to 40 mg, normally from 0.25 mg to 12 mg of pramipexole dihydrochloride monohydrate.

If said 6-propylamino-4,5,6,7-tetrahydro-1,3-benzothiazole-2-amine is pramipexole or a pharmaceutically acceptable salt or solvate thereof, said 6-propylamino-4,5,6,7-tetrahydro-1,3-benzothiazole-2-amine dose/unit form is equivalent to from 0.125 mg to 20 mg, from 0.375 to 12 mg, from 0.375 mg to 10 mg, from 0.375 mg to 7.5 mg, or from 0.375 mg to 6 mg, of pramipexole dihydrochloride monohydrate.

In particular embodiments, when said 6-propylamino-4,5,6,7-tetrahydro-1,3-benzothiazole-2-amine is pramipexole or a pharmaceutically acceptable salt or solvate thereof, it is present in a dose per unit form equivalent to from 0.125 mg to 6 mg, advantageously from 1.6 mg to 6 mg, preferably from 1.625 mg to 6 mg, of pramipexole dihydrochloride monohydrate. In these compositions, pramipexole may also be present in a dose per unit form equivalent to from 0.125 mg to 3 mg, advantageously from 1.6 mg to 3 mg, preferably from 1.625 mg to 3 mg, of pramipexole dihydrochloride monohydrate in an IR-formulation, or in a dose per unit form equivalent to from 1.5 mg to 6 mg, advantageously from 1.6 mg to 6 mg, preferably from 1.625 mg to 6 mg of pramipexole dihydrochloride monohydrate in an ER-formulation.

When said 6-propylamino-4,5,6,7-tetrahydro-1,3-benzothiazole-2-amine is a (S)/(R)-mixture in a fixed dose combination, said fixed-dose combination is a pharmaceutical composition in dosage unit form comprising said 6-propylamino-4,5,6,7-tetrahydro-1,3-benzothiazole-2-amine, in an amount per unit form equivalent to from 50 mg to 3000 mg of pramipexole dihydrochloride monohydrate, consisting of
i. a member selected from the group consisting of pramipexole and pharmaceutically acceptable salts and solvates thereof, in an amount equivalent to from 0.125 mg to 20 mg, normally from 0.125 mg to 6 mg, of pramipexole dihydrochloride monohydrate, and racemic 6-propylamino-4,5,6,7-tetrahydro-1,3-benzothiazole-2-amine and pharmaceutically acceptable salts and solvates thereof, in an amount equivalent to from 0.25 mg to 40 mg, normally from 0.25 mg to 12 mg of pramipexole dihydrochloride monohydrate; and
ii. (R)-6-propylamino-4,5,6,7-tetrahydro-1,3-benzothiazole-2-amine and pharmaceutically acceptable salts and solvates thereof, in an amount up to the total amount equivalent to from 50 mg to 3000 mg of pramipexole dihydrochloride monohydrate,
   in admixture with a pharmaceutical carrier or vehicle.

As stated in the Definitions, when generally citing the racemate or (R)/(S)-mixtures, the pramipexole included therein is referred to as (S)-enantiomer.

The above pharmaceutical compositions comprising said statin and said 6-propylamino-4,5,6,7-tetrahydro-1,3-benzothiazole-2-amine, preferably pramipexole, are in an unit form such as a tablet, a capsule, a pre-measured volume of a liquid solution or suspension for oral administration or a patch for transdermal application. Preferably, in said unit form, the statin and pramipexole or pharmaceutically acceptable salt thereof are formulated, separately or mixed together, in admixture with a pharmaceutical carrier or vehicle according to known technologies.

According to the method (or use) of the present invention, a statin, in combination with an effective daily dose of 6-propylamino-4,5,6,7-tetrahydro-1,3-benzothiazole-2-amine, is administered to a patient at a daily dose that is from half of the aforementioned daily dose approved for the treatment of dyslipidemia, up to the maximum daily dose approved for the treatment of dyslipidemia. Normally, as set forth above, the daily dose of said statin is from 0.5 to 80 mg.

The 6-propylamino-4,5,6,7-tetrahydro-1,3-benzothiazole-2-amine is administered to said patient, in combination with a statin, at a daily dose equivalent to from 0.375 mg to 3.000 mg of pramipexole dihydrochloride monohydrate, including a (S)-enantiomer daily dose equivalent to up to 20 mg, from 0.375 mg to 20 mg, normally from 0.375 mg to 6 mg, or at least from 0.375 mg to 4.5 mg of pramipexole dihydrochloride monohydrate.

If said 6-propylamino-4,5,6,7-tetrahydro-1,3-benzothiazole-2-amine is pramipexole or a pharmaceutically acceptable salt thereof, in combination with a daily dose of said statin of from 0.5 mg to 80 mg, it is administered at a daily dose equivalent to from 0.375 mg to 20 mg, from 0.375 to 15 mg, from 0.375 mg to 12 mg, from 0.375 mg to 10 mg, from 0.375 mg to 7.5 mg, or from 0.375 mg to 6 mg of pramipexole dihydrochloride monohydrate.

If, in said combination with said statin at the aforementioned daily dose, said 6-propylamino-4,5,6,7-tetrahydro-1,3-benzothiazole-2-amine is the above (S)/(R)-mixture in a fixed-dose combination comprising said Components (i) and (ii) wherein Component (i) is pramipexole or a pharmaceutically acceptable salt thereof, in an amount equivalent to from 0.125 to 20 mg of pramipexole dihydrochloride monohydrate, said (R)/(S)-mixture may be administered at a daily dose equivalent to from 150 mg to 3000 mg, normally from 300 mg to 3000 mg, including an (S)-enantiomer daily dose equivalent to from 0.375 mg to 20 mg of pramipexole dihydrochloride monohydrate. If said Component (i) is racemic propylamino-4,5,6,7-tetrahydro-1,3-benzothiazole-2-amine or a pharmaceutically acceptable salt thereof, in an amount equivalent to from 0.25 mg to 40 mg of pramipexole dihydrochloride monohydrate, said (R)/(S)-mixture is administered at a daily dose equivalent to from 150 mg to 3000 mg, normally from 300 mg to 3000 mg of pramipexole dihydrochloride monohydrate, including an (S)-enantiomer daily dose equivalent to up to 20 mg, from 0.375 mg to 20 mg, from 0.375 mg to 6 mg, or at least from 0.375 mg to 4.5 mg of pramipexole dihydrochloride monohydrate.

In some embodiments, if said 6-propylamino-4,5,6,7-tetrahydro-1,3-benzothiazole-2-amine is pramipexole, in combination with a daily dose of said statin of from 0.5 to 80 mg, said pramipexole is administered to said patient at a daily dose equivalent to from 0.375 mg to 6 mg, in particular from 1.5 to 6 mg, advantageously from 1.6 mg to 6 mg, preferably from 1.625 mg to 6 mg of pramipexole dihydrochloride monohydrate. If, in said combination with said statin at the aforementioned daily dose, said 6-propylamino-4,5,6,7-tetrahydro-1,3-benzothiazole-2-amine is the above (S)/(R)-mixture in a fixed-dose combination comprising said Components (i) and (ii) wherein Component (i) is pramipexole or a pharmaceutically acceptable salt thereof, in an amount equivalent to from 0.125 to 6 mg of pramipexole dihydrochloride monohydrate, said (R)/(S)-mixture is administered at a daily dose equivalent to from 150 mg to 3000 mg of pramipexole dihydrochloride monohydrate, normally from 300 mg to 3000 mg, including an (S)-enantiomer daily dose equivalent to up to 20 mg, from 0.375 mg to 6 mg, or from at least 0.375 mg to 4.5 mg of pramipexole dihydrochloride monohydrate. If said Component (i) is racemic propylamino-4,5,6,7-tetrahydro-1,3-benzothiazole-2-amine or a pharmaceutically acceptable salt thereof, in an amount equivalent to from 0.25 mg to 12 mg of pramipexole dihydrochloride monohydrate, said (R)/(S)-mixture is administered at a daily dose of from 150 mg to 3000 mg, normally from 300 mg to 3000 mg, including an (S)-enantiomer daily dose equivalent to from 0.75 mg to 12 mg of pramipexole dihydrochloride monohydrate.

Preferably, said daily dose of said statin is lower than the maximum daily dose approved for the treatment of dyslipidemia.

The present invention further provides a kit or package comprising a pharmaceutical combination or pharmaceutical composition as described herein, and instructions for use of the same for treatment of a synucleinopathy in a patient in need thereof.

### DETAILED DESCRIPTION

The present invention provides a pharmaceutical combination, including fixed-dose combinations, comprising a statin Component (a) and 6-propylamino-4,5,6,7-tetrahydro-1,3-benzothiazole-2-amine Component (b). This combination is useful for treating synucleinopathies such as PD, LBD, MSA, parkinsonian disorders associated with glucocerebrosidase (GBA) mutations, and others, in a patient in need of said treatment and, by consequence, the invention also provides
a method for the treatment of a synucleinopathy comprising administering an effective dose of a statin, in combination with 6-propylamino-4,5,6,7-tetrahydro-1,3-benzothiazole-2-amine;
a statin, for use for the treatment of a synucleinopathy in a patient, in combination, including fixed-dose combinations, with 6-propylamino-4,5,6,7-tetrahydro-1,3-benzothiazole-2-amine;
the use of a statin for the preparation of a medicament comprising said statin, as an active ingredient, for the treatment of a synucleinopathy, in combination with 6-propylamino-4,5,6,7-tetrahydro-1,3-benzothiazole-2-amine; and
a fixed dose combination comprising a pharmaceutical composition comprising a statin, as an active ingredient; and, as a second active ingredient, 6-propylamino-4,5,6,7-tetrahydro-1,3-benzothiazole-2-amine, in admixture with a pharmaceutical carrier or vehicle.

### The Statin

Several statins reportedly evidence neuroprotective activity in PD models, possibly due to anti-oxidant, anti-apoptotic, or anti-inflammatory mechanisms (Orr J D 2008), presumably as a consequence of reducing cholesterol via the mevalonate pathway (Saeedi Saravi S S et al. 2017).

This approach for the possible use of statins with neuroprotective intent was confirmed (Butterfield et al. 2011) even though, according to the authors, there was not strong enough clinical evidence to support the widespread use of statins to treat dementia and Alzheimer disease. These authors recommended further investigations.

Another statin, lovastatin, ameliorated alpha-synuclein accumulation and oxidation in transgenic mouse models of alpha-synucleinopathies (Koob A O et al. 2010).

Recently, rosuvastatin was found to have neuroprotective effect in SH-SY5Y cells against rotenone-induced neurotoxicity, as well as the modulation of α-synuclein expression (Kang S Y et al 2017).

Thus, prior to the present invention, the neuroprotective action of statins has not actually been evidenced in patients with synucleinopathies of the PD type and such action by statins alone would be expected to be minimal.

The statin is preferably selected from the group consisting of
(3R,5R)-7-[2-(4-fluorophenyl)-3-phenyl-4-(phenylcarbamoyl)-5-propan-2-ylpyrrol-1-yl]-3,5-dihydroxyheptanoic acid (atorvastatin) and pharmaceutically acceptable salts and solvates thereof, described in U.S. Pat. No. 5,273,995, the contents of which are incorporated herein in their entirety by reference;
(3R,5S,6E)-7-[3-(4-Fluorophenyl)-1-(propan-2-yl)-1H-indol-2-yl]-3,5-dihydroxyhept-6-enoic acid (fluvastatin), described in U.S. Pat. No. 4,739,073, the contents of which are incorporated herein in their entirety by reference;
(1S,3R,7S,8S,8aR)-8-{2-[(2R,4R)-4-Hydroxy-6-oxooxan-2-yl]ethyl}-3,7-dimethyl-1,2,3,7,8,8a-hexahydronaphthalen-1-yl (2S)-2-methylbutanoate (lovastatin), described in U.S. Pat. No. 4,231,938, the contents of which are incorporated herein in their entirety by reference;
(3R,5S,6E)-7-[2-Cyclopropyl-4-(4-fluorophenyl)quinolin-3-yl]-3,5-dihydroxyhept-6-enoic acid (pitavastatin) and pharmaceutically acceptable salts and solvates thereof, described in U.S. Pat. No. 5,011,930, the contents of which are incorporated herein in their entirety by reference;
(3R,5R)-3,5-dihydroxy-7-((1R,2S,6S,8R,8aR)-6-hydroxy-2-methyl-8-{[(2S)-2-methylbutanoyl]oxy}-1,2,6,7,8,8a-hexahydronaphthalen-1-yl)-heptanoic acid (pravastatin) and pharmaceutically acceptable salts and solvates thereof, described in U.S. Pat. No. 4,346,227, the contents of which are incorporated herein in their entirety by reference;
(1S,3R,7S,8S,8aR)-8-{2-[(2R,4R)-4-hydroxy-6-oxotetrahydro-2H-pyran-2-yl]ethyl}-3,7-dimethyl-1,2,3,7,8,8a-hexahydronaphthalen-1-yl 2,2-dimethylbutanoate; described in U.S. Pat. No. 4,444,784, the contents of which are incorporated herein in their entirety by reference; and
(3R,5S,6E)-7-[4-(4-Fluorophenyl)-2-(N-methylmethanesulfonamido)-6-(propan-2-yl)pyrimidin-5-yl]-3,5-dihydroxyhept-6-enoic acid (rosuvastatin) and pharmaceutically acceptable salts and solvates thereof, described in U.S. Pat. No. 5,260,440, the contents of which are incorporated herein in their entirety by reference.

Chemically, the known statins are characterized by a 3,5-dihydroxyheptane or 3,5-dihydroxyhept-6-ene carboxylic acid linked, via its 7-position, to a carbocyclic or heterocyclic structure. Thus, they can be in form of a lactone formed by loss of a H₂O between the carboxy group with the 5-hydroxy group of the 3,5-dihydroxyheptane carboxylic acid side-chain according to Scheme 1, wherein the steric configuration is not shown, and some of them are used in their lactone form.

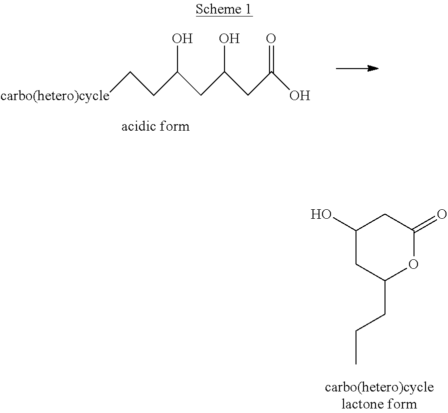

Both the acid and lactone forms of these acids are included in the family of statins of the present invention.

Herein, the expressions “salt or solvate thereof”, “salts or solvates thereof” and “salts and solvates thereof”, in reference to a statin in acidic form, indicate that the salt of said statin may be solvated with a solvent, normally water. Said salt normally is an alkaline metal salt or alkaline-earth metal salt, preferably sodium or calcium salt.

Advantageously, said stain is selected from the group consisting of atorvastatine and pharmaceutically acceptable salts and solvates thereof, fluvastatin and pharmaceutically acceptable salts and solvates thereof, lovastatin, pitavastatin and pharmaceutically acceptable salts and solvates thereof, pravastatin and pharmaceutically acceptable salts and solvates thereof, simvastatin, and rosuvastatin and pharmaceutically acceptable salts and solvates thereof.

A preferred statin is selected from the group consisting of atorvastatine calcium trihydrate, fluvastatin sodium, lovastatin, pitavastatin calcium, pravastatin sodium, simvastatin, and rosuvastatin calcium.

According to the present method, the statin is administered to said patient at a daily dose that is from the half of the aforementioned daily dose approved for the treatment of dyslipidemia, up to the maximum daily dose approved for the treatment of dyslipidemia. Normally, said statin is administered at a daily dose of from 0.5 mg to 80 mg. Preferably, said daily dose is lower than the maximum approved daily dose of each of said statins.

Preferably, in the treatment of a patient suffering from a synucleinopathy, in combination with an effective daily dose of 6-propylamino-4,5,6,7-tetrahydro-1,3-benzothiazole-2-amine, a statin selected from the group consisting of
atorvastatin calcium trihydrate, administered to said patient at a daily dose equivalent to from 5 mg to 80 mg, normally from 5 mg to 60 mg of atorvastatin free acid;
fluvastatin sodium, administered to said patient at a daily dose equivalent to from 10 mg to 80 mg, normally from 10 mg to 60 mg of fluvastatin free acid;
lovastatin, administered to said patient at a daily dose of from 5 mg to 80 mg, normally from 5 mg to 60 mg;
pitavastatin calcium, administered to said patient at a daily dose equivalent to from 0.5 mg to 4 mg, normally from 0.5 mg to 3 mg of pitavastatin free acid;
pravastatin sodium, administered to said patient at a daily dose of from 2.5 mg to 60 mg, normally from 2.5 mg to 40 mg;
simvastatin, administered to said patient at a daily dose of from 2.5 mg to 40 mg, normally from 2.5 mg to 30 mg; and
rosuvastatin calcium, administered to said patient at a daily dose of from 2.5 mg to 40 mg, normally from 2.5 mg to 30 mg, is particularly advantageous.

In order to be administered to a patient suffering from a synucleinopathy, the above statin is formulated in a pharmaceutical composition in dosage unit form comprising said statin in an amount per unit form of from 0.5 mg to 80 mg, in admixture with a pharmaceutical carrier or vehicle. Said statin is preferably selected from the group consisting of atorvastatine and pharmaceutically acceptable salts and solvates thereof, in an amount per unit form equivalent to from 5 mg to 80 mg, normally from 5 mg to 60 mg of atorvastatin free acid; fluvastatin and pharmaceutically acceptable salts and solvates thereof, in an amount per unit form equivalent to from 10 mg to 80 mg, normally from 10 mg to 60 mg of fluvastatin free acid; lovastatin, in an amount of from 5 mg to 80 mg, normally from 5 mg to 60 mg; pitavastatin and pharmaceutically acceptable salts and solvates thereof, in an amount per unit form equivalent to from 0.5 mg to 4 mg, normally from 0.5 mg to 3 mg of pitavastatin free acid; pravastatin and pharmaceutically acceptable salts and solvates thereof, in an amount per unit form equivalent to from 2.5 mg to 60 mg, normally from 2.5 mg to 40 mg of pravastatin sodium; simvastatin, in an amount per unit form of from 2.5 mg to 40 mg, normally from 2.5 mg to 30 mg; and rosuvastatin and pharmaceutically acceptable salts and solvates thereof, in an amount per unit form equivalent to from 2.5 mg to 40 mg, normally from 2.5 mg to 30 mg of rosuvastatin calcium.

### The 6-propylamino-4,5,6,7-tetrahydro-1,3-benzothiazole-2-amine

The 6-propylamino-4,5,6,7-tetrahydro-1,3-benzothiazole-2-amine is selected from the group consisting of
(S)-propylamino-4,5,6,7-tetrahydro-1,3-benzothiazole-2-amine (INN: pramipexole) and pharmaceutically acceptable salts and solvates thereof, in particular its dihydrochloride monohydrate (USAN: pramipexole hydrochloride), in a dose/unit form equivalent to from 0.125 mg to 20 mg, normally from 0.125 mg to 6 mg of pramipexole dihydrochloride monohydrate, administered in a daily dose equivalent to from 0.125 mg to 20 mg, normally from 0.375 mg to 6 mg of pramipexole dihydrochloride monohydrate;
a combination of
(i) (S)-propylamino-4,5,6,7-tetrahydro-1,3-benzothiazole-2-amine (INN: pramipexole) and pharmaceutically acceptable salts and solvates thereof, in a dose/unit form equivalent to from 0.125 mg to 20 mg. normally from 0.125 mg to 6 mg of pramipexole dihydrochloride monohydrate, administered in a daily dose equivalent to up to 20 mg, from 0.375 mg to 20 mg, normally from 0.375 mg to 6 mg, or from at least 0.375 mg to 4.5 mg of pramipexole dihydrochloride monohydrate; and
(ii) (R)-propylamino-4,5,6,7-tetrahydro-1,3-benzothiazole-2-amine and pharmaceutically acceptable salts and solvates thereof, in a dose/unit form equivalent to from 50 mg to 3000 mg of dexpramipexole dihydrochloride monohydrate, concurrently or sequentially administered with Component (i), in a daily dose (in dexpramipexole dihydrochloride monohydrate) of from 150 mg to 3000 mg; and
a (S)/(R)-mixture (fixed-dose combination) that is a pharmaceutical composition in dosage unit form comprising a 6-propylamino-4,5,6,7-tetrahydro-1,3-benzothiazole-2-amine, in a dose per unit form of from 50 mg to 3000 mg, said dose per unit form including
(i) a pramipexole, or pharmaceutically acceptable salts or solvate thereof dose per unit form equivalent to from 0.125 mg to 20 mg, normally from 0.125 mg to 6 mg of pramipexole dihydrochloride monohydrate, or a racemate, or pharmaceutically acceptable salt or solvate thereof dose per unit form equivalent to from 0.25 mg to 40 mg, normally from 0.25 mg to 12 mg of pramipexole dihydrochloride monohydrate; and
(ii) a (R)-6-propylamino-4,5,6,7-tetrahydro-1,3-benzothiazole-2-amine, or a pharmaceutically acceptable salt thereof dose per unit form up to the total dose per unit form equivalent of 3000 mg of pramipexole dihydrochloride monohydrate, in admixture with a pharmaceutical carrier or vehicle.

In order to be administered to a patient suffering from a synucleinopathy in combination with a daily dose of from 0.5 mg to 80 mg of said statin, said 6-propylamino-4,5,6,7-tetrahydro-1,3-benzothiazole-2-amine, as pramipexole, as dexpramipexole, as racemate, as (S)-(R)-combination, or as (R)/(S)-mixture, including fixed-dose combinations, is formulated in a pharmaceutical composition in dosage unit from comprising the aforementioned, respective dose-range per unit form of each of them, each in admixture with a pharmaceutical carrier or vehicle.

Pharmaceutical compositions comprising (R)-6-propylamino-4,5,6,7-tetrahydro-1,3-benzothiazole-2-amine or pharmaceutically acceptable salt thereof are disclosed in US 2013/0116292, the contents of which are incorporated herein in their entirety by reference.

Racemic 6-propylamino-4,5,6,7-tetrahydro-1,3-benzothiazole-2-amine and its resolution have been described by Schneider C S and Mierau J, (1987), and also in U.S. Pat. No. 7,285,669, the contents of which are incorporated herein in their entirety by reference.

(S)-(R)-combinations and (S)/(R)-mixtures, consisting of pharmaceutical compositions comprising a therapeutically effective amount of (R)-6-propylamino-4,5,6,7-tetrahydro-1,3-benzothiazole-2-amine or pharmaceutically acceptable salts and solvates thereof and a therapeutically effective amount of (S)-6-propylamino-4,5,6,7-tetrahydro-1,3-benzothiazole-2-amine or pharmaceutically acceptable salts and solvates thereof, are disclosed in US 2008/0014259 (U.S. Pat. No. 8,017,598), the contents of which are incorporated herein in their entirety by reference.

In combination with a statin, 6-propylamino-4,5,6,7-tetrahydro-1,3-benzothiazole-2-amine, as racemate, as pramipexole, as (S)-(R)-combination or as (S)/(R)-mixture may be administered, daily or per unit form doses (in pramipexole or (S)-enantiomer) not only as high as those approved for pramipexole or disclosed for known mixtures, but also at higher doses (daily or per unit form).

Pharmaceutically acceptable salts of 6-propylamino-4,5,6,7-tetrahydro-1,3-benzothiazole-2-amine are also included in the present invention. Illustrative examples of these salts include acid addition salts with mineral or organic acids such as hydrochloric acid, hydrobromic acid, hydriodic acid, sulfuric acid, sulfamic acid, nitric acid, phosphoric acid, formic acid, acetic acid, propionic acid, stearic acid, glycolic acid, oxalic acid, malonic acid, succinic acid, fumaric acid, maleic acid, hydroxymaleic acid, malonic acid, lactic acid, malic acid, tartaric acid, citric acid, carbonic acid, ascorbic acid, phenylacetic acid, benzoic acid, salicylic acid, 2-acetoxybenzoic acid, methanesulfonic acid, ethanesulfonic acid, 2-hydroxyethanesulfonic (isethionic) acid, p-toluenesulfonic acid, 2-naphthalenesulfonic acid, 4-amino-benzenesulfonic (sulfanilic) acid, 2,6-naphthalenedisulfonic acid, 1,5-naphthalenedisulfonic acid, pamoic (embonic) acid, aspartic acid, glutamic acid and the like. The solvation agent is generally water.

According to a preferred embodiment, in the treatment of a patient suffering from a synucleinopathy and in combination with a statin, the 6-propylamino-4,5,6,7-tetrahydro-1,3-benzothiazole-2-amine is selected from the group consisting of (S)-propylamino-4,5,6,7-tetrahydro-1,3-benzothiazole-2-amine (pramipexole) and pharmaceutically acceptable salts and solvates thereof, in a daily dose equivalent to from 0.375 mg to 20 mg or from 0.375 mg to 6 mg of pramipexole dihydrochloride monohydrate.

According to this preferred embodiment, said statin is selected from the group consisting of atorvastatine and pharmaceutically acceptable salts and solvates thereof, in an amount per unit form equivalent to from 5 mg to 80 mg, normally from 5 mg to 60 mg of atorvastatin free acid, administered to said patient at a daily dose equivalent to from 5 mg to 80 mg, normally from 5 mg to 60 mg of atorvastatin free acid; fluvastatin and pharmaceutically acceptable salts and solvates thereof, in an amount per unit form equivalent to from 10 mg to 80 mg, normally from 10 mg to 60 mg of fluvastatin free acid, administered to said patient at a daily dose equivalent to from 10 mg to 80 mg, normally from 10 mg to 60 mg of fluvastatin free acid; lovastatin, in an amount of from 5 mg to 80 mg, normally from 5 mg to 60 mg, administered to said patient at a daily dose of from 5 mg to 80 mg, normally from 5 mg to 60 mg; pitavastatin and pharmaceutically acceptable salts and solvates thereof, in an amount per unit form equivalent to from 0.5 mg to 4 mg, normally from 0.5 mg to 3 mg of pitavastatin free acid, administered to said patient at a daily dose equivalent to from 0.5 mg to 4 mg, normally from 0.5 mg to 3 mg of pitavastatin free acid; pravastatin and pharmaceutically acceptable salts and solvates thereof, in an amount per unit form equivalent to from 2.5 mg to 60 mg, normally from 2.5 mg to 40 mg of pravastatin sodium, administered to said patient at a daily dose of from 2.5 mg to 60 mg, normally from 2.5 mg to 40 mg; simvastatin, in an amount per unit form of from 2.5 mg to 40 mg, normally from 2.5 mg to 30 mg, administered to said patient at a daily dose of from 2.5 mg to 40 mg, normally from 2.5 mg to 30 mg; and rosuvastatin and pharmaceutically acceptable salts and solvates thereof, in an amount per unit form equivalent to from 2.5 mg to 40 mg, normally from 2.5 mg to 30 mg of rosuvastatin calcium, administered to said patient at a daily dose of from 2.5 mg to 40 mg, normally from 2.5 mg to 30 mg.

### Specific Aspects of the Invention

According to specific aspects, the invention provides a pharmaceutical combination comprising a statin Component (a) and 6-propylamino-4,5,6,7-tetrahydro-1,3-benzothiazole-2-amine Component (b), for use in the treatment of a synucleinopathy, in particular in a patient suffering from Parkinson's disease, Lewy body dementia (LBD), dementia with Lewy bodies (DLB), Alzheimer's disease (AD), the Lewy body variant of AD, multiple system atrophy, neurodegeneration with brain iron accumulation, and parkinsonian disorders associated with glucocerebrosidase (GBA) mutations.

### First Aspect of the Invention

According to a-first aspect, the invention provides a method for treating a patient suffering from a synucleinopathy, which comprises treating said patient with an effective daily dose of a statin, in combination with an effective daily dose of 6-propylamino-4,5,6,7-tetrahydro-1,3-benzothiazole-2-amine or of a pharmaceutically acceptable salt or solvate thereof.

Herein below, the expressions “salt or solvate thereof”, “salts or solvates thereof” and “salts and solvates thereof”, in reference to said 6-propylamino-4,5,6,7-tetrahydro-1,3-benzothiazole-2-amine mean that said 6-propylamino-4,5,6,7-tetrahydro-1,3-benzothiazole-2-amine may be in the form of the free base or of a pharmaceutically acceptable acid addition salt thereof that may be solvated with a solvent, normally water.

In the method according to the present invention, 6-propylamino-4,5,6,7-tetrahydro-1,3-benzothiazole-2-amine or pharmaceutically acceptable salt or solvate thereof is administered to a patient suffering from a synucleinopathy at a daily dose equivalent to from 0.375 mg to 3000 mg of pramipexole dihydrochloride monohydrate, said dose including a (S)-enantiomer daily dose equivalent to up to 20 mg, from 0.375 mg to 20 mg, normally from 0.375 mg to 6 mg, or from at least 0.375 mg to 4.5 mg of pramipexole dihydrochloride monohydrate, in combination with a statin.

In particular, when said 6-propylamino-4,5,6,7-tetrahydro-1,3-benzothiazole-2-amine is a (S)/(R) fixed-dose combination, it is administered to said patient, in combination with a statin daily dose of from 0.5 to 80 mg, at a daily dose equivalent to from 150 mg to 3000 mg, or from 300 mg to 3000 mg, of pramipexole dihydrochloride monohydrate. Said daily dose includes a (S)-enantiomer daily dose equivalent to up to 20 mg, from 0.375 mg to 20 mg, from 0.375 to 15 mg, from 0.375 mg to 12 mg, from 0.375 mg to 10 mg, from 0.375 mg to 7.5 mg, from 0.375 mg to 6 mg, or at least from 0.375 mg to 4.5 mg, advantageously from 1.6 mg to 6 mg, preferably from 1.625 mg to 6 mg of pramipexole dihydrochloride monohydrate.

In order to be administered to a patient suffering from a synucleinopathy, at a daily dose of from 0.5 mg to 80 mg, the above statin is formulated in a pharmaceutical composition in dosage unit from comprising said statin in an amount per unit form of from 0.5 mg to 80 mg, in admixture with a pharmaceutical carrier or vehicle, said statin being preferably selected from the group consisting of those listed above in “The Statin” section, each at the dose per unit form and at the daily dose described therein.

Said statin, in said amount per unit form, is administered to said patient suffering from a synucleinopathy, in combination with 6-propylamino-4,5,6,7-tetrahydro-1,3-benzothiazole-2-amine, said 6-propylamino-4,5,6,7-tetrahydro-1,3-benzothiazole-2-amine being preferably pramipexole or a pharmaceutically acceptable salt or solvate thereof, in an amount per unit form equivalent to from 0.125 mg to 20 mg, from 0.125 to 15 mg, from 0.125 mg to 12 mg, from 0.125 mg to 10 mg, from 0.125 mg to 7.5 mg, from 0.125 mg to 6 mg, from 1.6 mg to 6 mg, from 1.625 mg to 6 mg or from 3 mg to 6 mg of pramipexole dihydrochloride monohydrate.

According to an advantageous embodiment,

said statin is administered to said patient in a pharmaceutical composition in dosage unit form comprising said statin, selected from the group consisting of atorvastatine and pharmaceutically acceptable salts and solvates thereof, in an amount per unit form equivalent to from 5 mg to 80 mg of atorvastatin free acid; fluvastatin and pharmaceutically acceptable salts and solvates thereof, in an amount per unit form equivalent to from 10 mg to 80 mg of fluvastatin free acid; lovastatin, in an amount per unit of from 5 mg to 80 mg; pitavastatin and pharmaceutically acceptable salts and solvates thereof, in an amount per unit form equivalent to from 0.5 mg to 4 mg of pitavastatin free acid; pravastatin and pharmaceutically acceptable salts and solvates thereof, in an amount per unit form equivalent to from 2.5 mg to 60 mg of pravastatin sodium; simvastatin, in an amount per unit form of from 2.5 mg to 40 mg; and rosuvastatin and pharmaceutically acceptable salts and solvates thereof, in an amount per unit form equivalent to from 2.5 mg to 40 mg of rosuvastatin calcium,

in admixture with a pharmaceutical carrier or vehicle; and

said 6-propylamino-4,5,6,7-tetrahydro-1,3-benzothiazole-2-amine is administered to said patient in a pharmaceutical composition in dosage unit form comprising said 6-propylamino-4,5,6,7-tetrahydro-1,3-benzothiazole-2-amine, selected from the group consisting of pramipexole and pharmaceutically acceptable salts and solvates thereof, in an amount per unit form equivalent to a range selected from the group consisting of from 0.125 mg to 20 mg, from 0.125 to 15 mg, from 0.125 mg to 12 mg, from 0.125 mg to 10 mg, from 0.125 mg to 7.5 mg, from 0.125 mg to 6 mg, from 1.6 mg to 6 mg, from 1.625 mg to 6 mg, and from 3 mg to 6 mg of pramipexole dihydrochloride monohydrate, in admixture with a pharmaceutical carrier or vehicle.

According to a preferred embodiment, said statin and said 6-propylamino-4,5,6,7-tetrahydro-1,3-benzothiazole-2-amine are concurrently administered to said patient in a fixed-dose combination, in a dosage unit form wherein said statin active ingredient and said 6-propylamino-4,5,6,7-tetrahydro-1,3-benzothiazole-2-amine are compounded together or separately in said unit form. Said fixed-dose combination are described below in “The fourth aspect of the invention” and in “The formulations” sections.

### Second Aspect of the Invention

According to a second aspect, the invention provides a statin, for use in the treatment of a synucleinopathy in a patient in need of said treatment, in combination with an effective daily dose of 6-propylamino-4,5,6,7-tetrahydro-1,3-benzothiazole-2-amine.

The use according to this second aspect of the present invention includes the administration of said statin and said 6-propylamino-4,5,6,7-tetrahydro-1,3-benzothiazole-2-amine in the respective effective dose per unit form, under the conditions and the respective statin and 6-propylamino-4,5,6,7-tetrahydro-1,3-benzothiazole-2-amine daily doses according to the method of the above first aspect of the invention.

For said administration to a patient suffering from a synucleinopathy, the statin is formulated in a pharmaceutical composition in dosage unit form comprising said statin in an amount per unit form of from 0.5 mg to 80 mg, in admixture with a pharmaceutical carrier or vehicle.

The pharmaceutical composition is administered to said patient in combination with a 6-propylamino-4,5,6,7-tetrahydro-1,3-benzothiazole-2-amine, also in a pharmaceutical composition in dosage unit form comprising said 6-propylamino-4,5,6,7-tetrahydro-1,3-benzothiazole-2-amine in an amount per unit form equivalent to from 0.125 mg to 3000 mg of pramipexole dihydrochloride monohydrate, including a (S)-enantiomer amount per unit form equivalent to from 0.125 mg to 20 mg of pramipexole dihydrochloride monohydrate, in admixture with a pharmaceutical carrier.

Said statin is preferably selected from the group consisting of atorvastatine and pharmaceutically acceptable salts and solvates thereof, in an amount per unit form equivalent to from 5 mg to 80 mg, normally from 5 mg to 60 mg of atorvastatin free acid, administered to said patient at a daily dose equivalent to from 5 mg to 80 mg, normally from 5 mg to 60 mg of atorvastatin free acid; fluvastatin and pharmaceutically acceptable salts and solvates thereof, in an amount per unit form equivalent to from 10 mg to 80 mg, normally from 10 mg to 60 mg of fluvastatin free acid, administered to said patient at a daily dose equivalent to from 10 mg to 80 mg, normally from 10 mg to 60 mg of fluvastatin free acid; lovastatin, in an amount per unit form of from 5 mg to 80 mg, normally from 5 mg to 60 mg, administered to said patient at a daily dose of from 5 mg to 80 mg, normally from 5 mg to 60 mg; pitavastatin and pharmaceutically acceptable salts and solvates thereof, in an amount per unit form equivalent to from 0.5 mg to 4 mg, normally from 0.5 mg to 3 mg of pitavastatin free acid, administered to said patient at a daily dose equivalent to from 0.5 mg to 4 mg, normally from 0.5 mg to 3 mg of pitavastatin free acid; pravastatin and pharmaceutically acceptable salts and solvates thereof, in an amount per unit form equivalent to from 2.5 mg to 60 mg, normally from 2.5 mg to 40 mg of pravastatin sodium, administered to said patient at a daily dose of equivalent to from 2.5 mg to 60 mg, normally from 2.5 mg to 40 mg of pravastatin sodium; simvastatin, in an amount per unit form of from 2.5 mg to 40 mg, normally from 2.5 mg to 30 mg, administered to said patient at a daily dose of from 2.5 mg to 40 mg, normally from 2.5 mg to 30 mg; and rosuvastatin and pharmaceutically acceptable salts and solvates thereof, in an amount per unit form equivalent to from 2.5 mg to 40 mg, normally from 2.5 mg to 30 mg of rosuvastatin calcium, administered to said patient at a daily dose equivalent to from 2.5 mg to 40 mg, normally from 2.5 mg to 30 mg of rosuvastatin calcium.

Said statin in said dose per unit form is administered at said daily dose in combination with 6-propylamino-4,5,6,7-tetrahydro-1,3-benzothiazole-2-amine, said 6-propylamino-4,5,6,7-tetrahydro-1,3-benzothiazole-2-amine being preferably selected from the group consisting of pramipexole and pharmaceutically acceptable salts and solvates thereof, in an amount per unit form equivalent to from 0.125 mg to 20 mg, from 0.125 to 15 mg, from 0.125 mg to 12 mg, from 0.125 mg to 10 mg, from 0.125 mg to 7.5 mg, from 0.125 mg to 6 mg, from 1.6 mg to 6 mg, from 1.625 mg to 6 mg, or from 3 mg to 6 mg of pramipexole dihydrochloride monohydrate, at a daily dose equivalent to from 0.375 mg to 20 mg, from 0.375 to 15 mg, from 0.375 mg to 12 mg, from 0.375 mg to 10 mg, from 0.375 mg to 7.5 mg, from 0.375 mg to 6 mg, from 1.6 mg to 6 mg, from 1.625 mg to 6 mg, or from 3 mg to 6 mg of pramipexole dihydrochloride monohydrate.

### Third Aspect of the Invention

According to a third aspect, the invention provides the use of a statin for the preparation of a medicament for the treatment of a synucleinopathy in a patient in need of said treatment, in combination with an effective daily dose of 6-propylamino-4,5,6,7-tetrahydro-1,3-benzothiazole-2-amine.

In particular, said medicament is a pharmaceutical composition in dosage unit form comprising said statin in a dose per unit form of from the half the minimum dose to the maximum dose per unit form approved for the treatment of a dyslipidemia.

This third aspect of the present invention includes the manufacture of a medicament consisting of a statin, in a pharmaceutical composition in dosage unit form comprising said statin, in a dose/unit form of from 0.5 mg to 80 mg, in admixture with a pharmaceutical carrier or vehicle, for the treatment of a synucleinopathy in a patient, in combination with an effective daily dose of 6-propylamino-4,5,6,7-tetrahydro-1,3-benzothiazole-2-amine.

More particularly, in said composition, said statin is administered at a daily dose of from 0.5 mg to 80 mg and said 6-propylamino-4,5,6,7-tetrahydro-1,3-benzothiazole-2-amine, also in a pharmaceutical composition, is administered at a daily dose equivalent to from 0.375 mg to 3000 mg of pramipexole dihydrochloride monohydrate, including a (S)-enantiomer daily dose equivalent to from 0.375 mg to 20 mg, normally from 0.375 mg to 6 mg of pramipexole dihydrochloride monohydrate.

If said 6-propylamino-4,5,6,7-tetrahydro-1,3-benzothiazole-2-amine is pramipexole or a pharmaceutically acceptable salt thereof, it is administered at a daily dose equivalent to from 0.375 mg to 20 mg, from 0.375 to 15 mg, from 0.375 mg to 12 mg, from 0.375 mg to 10 mg, from 0.375 mg to 7.5 mg, from 0.375 to 6 mg, from 1.6 mg to 6 mg, from 1.625 mg to 6 mg, or from 3 mg to 6 mg of pramipexole dihydrochloride monohydrate.

According to this third aspect of the present invention, said statin is manufactured in a pharmaceutical composition in dosage unit form comprising, as an active ingredient, said statin, in an amount per unit form of from 0.5 mg to 80 mg, in admixture with a pharmaceutical carrier or vehicle, to be administered in combination with 6-propylamino-4,5,6,7-tetrahydro-1,3-benzothiazole-2-amine, also in a pharmaceutical composition comprising said 6-propylamino-4,5,6,7-tetrahydro-1,3-benzothiazole-2-amine in an amount equivalent to from 0.125 mg to 3000 mg of pramipexole dihydrochloride monohydrate, including a (S)-enantiomer amount per unit form of from 0.125 mg to 20 mg, normally from 0.125 mg to 6 mg of pramipexole dihydrochloride monohydrate, in admixture with a pharmaceutical carrier or vehicle. If said 6-propylamino-4,5,6,7-tetrahydro-1,3-benzothiazole-2-amine is pramipexole or a pharmaceutically acceptable salt thereof, it is present in said composition in an amount per unit form equivalent to from 0.125 mg to 20 mg, from 0.125 to 15 mg, from 0.125 mg to 12 mg, from 0.125 mg to 10 mg, from 0.125 mg to 7.5 mg, from 0.125 mg to 6 mg, from 1.6 mg to 6 mg from 1.625 mg to 6 mg, or from 3 mg to 6 mg of pramipexole dihydrochloride monohydrate.

Preferably, said pharmaceutical composition in dosage unit form comprises a statin selected from the group consisting of atorvastatin calcium trihydrate, in an amount of from 5 mg to 80 mg, preferably from 5 mg to 60 mg; fluvastatin sodium, in an amount of from 10 mg to 80 mg, preferably from 10 mg to 60 mg; lovastatin, in an amount of from 10 mg to 40 mg, preferably from 10 mg to 30 mg; pitavastatin calcium, in an amount of from 0.5 mg to 4 mg, preferably from 0.5 mg to 3 mg; pravastatin sodium, in an amount of from 5 mg to 80 mg, preferably from 5 mg to 60 mg; simvastatin, in an amount of from 2.5 mg to 80 mg, preferably from 2.5 mg to 60 mg; and rosuvastatin calcium, in an amount of from 2.5 mg to 40 mg, preferably from 2.5 mg to 30 mg, in admixture with a pharmaceutical carrier or vehicle. This composition is administered to a patient suffering from a synucleinopathic disorder, in combination with an effective daily dose of 6-propylamino-4,5,6,7-tetrahydro-1,3-benzothiazole-2-amine, also in a pharmaceutical composition in dosage unit form comprising said 6-propylamino-4,5,6,7-tetrahydro-1,3-benzothiazole-2-amine in an amount per unit form equivalent to from 0.125 mg to 3000 mg of pramipexole dihydrochloride monohydrate, including a (S)-enantiomer amount per unit form equivalent to from 0.125 mg to 20 mg of pramipexole dihydrochloride monohydrate, in admixture with a pharmaceutical carrier or vehicle.

Preferably, said 6-propylamino-4,5,6,7-tetrahydro-1,3-benzothiazole-2-amine is pramipexole or a pharmaceutically acceptable salt or solvate thereof, in a dose/unit form equivalent to from 0.125 mg to 20 mg, from 0.125 to 15 mg, from 0.125 mg to 12 mg, from 0.125 mg to 10 mg, from 0.125 mg to 7.5 mg, from 0.125 to 6 mg, from 1.6 mg to 6 mg, from 1.625 mg to 6 mg, or from 3 mg to 6 mg of pramipexole dihydrochloride monohydrate. It is administered to said patient at a daily dose equivalent to from 0.375 mg to 20 mg, normally from 0.375 mg to 6 mg of pramipexole dihydrochloride monohydrate.

The statin and the 6-propylamino-4,5,6,7-tetrahydro-1,3-benzothiazole-2-amine can be administered separately or together in any conventional oral or parenteral dosage unit form such as capsule, tablet, powder, cachet, suspension, solution, or transdermal device.

In the case of separate (concurrent or sequential) administration of said statin, in an effective amount per unit form, and said 6-propylamino-4,5,6,7-tetrahydro-1,3-benzothiazole-2-amine, in an effective amount per unit form, each of them can be packaged in a kit comprising said statin, in admixture with a pharmaceutical carrier or vehicle, in a container; and said 6-propylamino-4,5,6,7-tetrahydro-1,3-benzothiazole-2-amine, preferably pramipexole, in admixture with a pharmaceutical carrier or vehicle, in another, separate container.

For their concurrent administration for the treatment of synucleinopathies, said statin and said 6-propylamino-4,5,6,7-tetrahydro-1,3-benzothiazole-2-amine or a pharmaceutically acceptable salt or solvate thereof may also be formulated together in a fixed-dose combination consisting of a pharmaceutical composition comprising said statin and said 6-propylamino-4,5,6,7-tetrahydro-1,3-benzothiazole-2-amine, in admixture with a pharmaceutical carrier or vehicle.

In particular, this third aspect of the invention also provides the use of a statin for the preparation of a medicament for the treatment of a synucleinopathy, consisting of a pharmaceutical composition in dosage unit form comprising, as an active ingredient, said statin, and, as another active ingredient, said 6-propylamino-4,5,6,7-tetrahydro-1,3-benzothiazole-2-amine, in admixture with a pharmaceutical carrier or vehicle.

Said medicament, i.e. said pharmaceutical composition, in the effective doses per unit form and at the effective daily doses for the treatment of a synucleinopathy will be described in the “Fourth aspect of the invention” and in “The formulations” sections below.

### Fourth Aspect of the Invention

As mentioned above, in carrying out the method (or use) according to a preferred embodiment of present invention, said statin and said 6-propylamino-4,5,6,7-tetrahydro-1,3-benzothiazole-2-amine are concurrently administered to said patient in a fixed-dose combination, in a single dosage unit form, wherein said statin active ingredient and said 6-propylamino-4,5,6,7-tetrahydro-1,3-benzothiazole-2-amine active ingredient are compounded together or separately, in said single unit form and in admixture with a pharmaceutical carrier or vehicle.

According to a fourth aspect, the invention provides a fixed-dose combination consisting of a pharmaceutical composition in dosage unit form comprising, as Components,

Component (a) a statin; and
Component (b) 6-propylamino-4,5,6,7-tetrahydro-1,3-benzothiazole-2-amine; in admixture with a pharmaceutically acceptable carrier or vehicle.

As set forth above, the statin Component (a) dose per unit form is from the half of the minimum dose approved for the treatment of dyslipidemia to the maximum dose approved for the treatment of dyslipidemia, but in a preferred embodiment, the maximum amount of the dose range of the statin Component (a) is lower than that of said statin as approved for the treatment of dyslipidemia.

Thus, according to this fourth aspect, the present invention provides the use of a statin Component (a) for the preparation of a medicament for the treatment of a synucleinopathy, said medicament consisting of a dosage unit form comprising said statin, as an active ingredient, in an amount per unit form and, as a second active ingredient, 6-propylamino-4,5,6,7-tetrahydro-1,3-benzothiazole-2-amine Component (b), in an amount per unit form, in pramipexole dihydrochloride monohydrate, of from 0.125 mg to 3000 mg, including a (S)-enantiomer amount per unit form equivalent to from 0.125 mg to 20 mg, normally from 0.125 mg to 6 mg, of pramipexole dihydrochloride monohydrate, formulated in admixture with a pharmaceutical carrier or vehicle.

Said use will be described in “The formulations” section below.

The dose of the 6-propylamino-4,5,6,7-tetrahydro-1,3-benzothiazole-2-amine per IR-unit form, in pramipexole dihydrochloride monohydrate, will range from 0.125 mg to 1500 mg, in particular from 1.5 mg to 1500 mg, from 1.6 mg to 1500 mg, depending on safety and tolerability (in the fixed-dose combination with the statin), said dose per unit form including a (S)-enantiomer amount equivalent to from 0.125 mg to 10 mg of pramipexole dihydrochloride monohydrate.

If said 6-propylamino-4,5,6,7-tetrahydro-1,3-benzothiazole-2-amine is pramipexole or a pharmaceutically acceptable salt thereof, the dose-range per IR-unit form will be equivalent to from 0.125 mg to 10 mg, from 0.125 to 7.5 mg, from 0.125 mg to 6 mg, from 0.125 mg to 5 mg, from 0.125 mg to 3.75 mg, from 0.125 mg to 3 mg, from 0.125 mg to 1.5 mg, from 0.125 mg to 0.75 mg, or from 0.125 mg to 0.375 mg, normally from 1.5 mg to 3 mg, from 1.6 mg to 3 mg, or from-1.625 mg to 3 mg of pramipexole dihydrochloride monohydrate, depending on safety and tolerability (in the fixed-dose combination with the statin).

The dose per unit form of 6-propylamino-4,5,6,7-tetrahydro-1,3-benzothiazole-2-amine in an ER formulation, including slow-release compositions and 6-propylamino-4,5,6,7-tetrahydro-1,3-benzothiazole-2-amine released from transdermal therapeutic systems such as transdermal patches, will range (in pramipexole dihydrochloride monohydrate) from 1.5 mg to 3000 mg, normally from 1.6 to 3000 mg, advantageously, from 3 mg to 3000 mg, depending on the tolerability (in the fixed-dose combination with the statin), said dose per unit form including a (S)-enantiomer amount per unit form equivalent to from 0.375 mg to 20 mg, normally from 0.375 mg to 6 mg, of pramipexole dihydrochloride monohydrate.

If said 6-propylamino-4,5,6,7-tetrahydro-1,3-benzothiazole-2-amine is pramipexole or a pharmaceutically acceptable salt thereof, the dose range per ER-unit form will be equivalent to from 0.375 mg to 20 mg, from 0.375 to 15 mg, from 0.375 mg to 12 mg, from 0.375 mg to 10 mg, from 0.375 mg to 7.5 mg, or from 0.375 mg to 6 mg of pramipexole dihydrochloride monohydrate, preferably equivalent to 0.375 mg, 0.75 mg, 1.5 mg, 1.6 mg, 2.25 mg, 3 mg, 3.75 mg, or 4.5 mg or to more than 4.5 mg to 6 mg of pramipexole dihydrochloride monohydrate, more preferably equivalent to from 1.6 mg to 6 mg, from 3 mg to 6 mg, or most preferably equivalent to from more than 4.5 mg to 6 mg, of pramipexole dihydrochloride monohydrate.

According to an advantageous embodiment, the invention provides a pharmaceutical composition in dosage unit form comprising, as Components,
Component (a) a statin selected from the group consisting of atorvastatin calcium trihydrate, in an amount of from 5 mg to 80 mg, preferably from 5 mg to 60 mg; fluvastatin sodium, in an amount of from 10 mg to 80 mg, preferably from 10 mg to 60 mg; lovastatin, in an amount of from 10 mg to 40 mg, preferably from 10 mg to 30 mg; pitavastatin calcium, in an amount of from 0.5 mg to 4 mg, preferably from 0.5 mg to 3 mg; pravastatin sodium, in an amount of from 5 mg to 80 mg, preferably from 5 mg to 60 mg; simvastatin, in an amount of from 2.5 mg to 80 mg, preferably from 2.5 mg to 60 mg; and rosuvastatin calcium, in an amount of from 2.5 mg to 40 mg, preferably from 2.5 mg to 30 mg; and
Component (b) 6-propylamino-4,5,6,7-tetrahydro-1,3-benzothiazole-2-amine, in an amount per unit form equivalent to from 0.125 mg to 3000 mg, advantageously from 1.6 mg to 3000 mg, preferably from 1.625 mg to 3000 mg of pramipexole dihydrochloride monohydrate, including a (S)-enantiomer amount equivalent to from 0.125 mg to 20 mg of pramipexole dihydrochloride monohydrate,
   in admixture with a pharmaceutical carrier or vehicle.

In particular, said Component (b) may be a (S)/(R)-mixture consisting of
(i) a member selected from the group consisting of
   pramipexole and pharmaceutically acceptable salts and solvates thereof, in a dose per unit form equivalent to from 0.125 mg to 20 mg, normally from 0.125 mg to 6 mg of pramipexole dihydrochloride monohydrate; and
   racemic 6-propylamino-4,5,6,7-tetrahydro-1,3-benzothiazole-2-amine and pharmaceutically acceptable salt and solvates thereof, in an amount per unit form equivalent to from 0.25 mg to 40 mg, normally from 0.25 mg to 12 mg of pramipexole dihydrochloride monohydrate, and
(ii) (R)-6-propylamino-4,5,6,7-tetrahydro-1,3-benzothiazole-2-amine or a pharmaceutically acceptable salt thereof, in an amount per unit form up to the total (in pramipexole dihydrochloride monohydrate) of from 50 mg to 3000 mg.

More particularly, said 6-propylamino-4,5,6,7-tetrahydro-1,3-benzothiazole-2-amine Component (b) may be a mixture of
(i) (S)-6-propylamino-4,5,6,7-tetrahydro-1,3-benzothiazole-2-amine or a pharmaceutically acceptable salt thereof, in a dose/unit form equivalent to from 0.125 mg to 20 mg, normally from 0.125 mg to 6 mg of pramipexole dihydrochloride monohydrate; and
(ii) (R)-6-propylamino-4,5,6,7-tetrahydro-1,3-benzothiazole-2-amine or a pharmaceutically acceptable salts thereof, in a dose/unit form up to a total (in pramipexole dihydrochloride monohydrate) of from 50 mg to 3000 mg,
   in admixture with a pharmaceutical carrier or vehicle.

Preferably, a dosage unit form, formulated in admixture with a pharmaceutical carrier or vehicle, comprises rosuvastatin or a pharmaceutically acceptable salt or solvate thereof, as active ingredient Component (a), in an amount per unit form equivalent to from 2.5 mg to 40 mg of rosuvastatin calcium; and, as a second active ingredient Component (b), pramipexole or a pharmaceutically acceptable salt or solvate thereof, in an amount per unit form equivalent to from 0.125 mg to 20 mg or from 0.125 mg to 6 mg of pramipexole dihydrochloride monohydrate.

More particularly, said 6-propylamino-4,5,6,7-tetrahydro-1,3-benzothiazole-2-amine is pramipexole or a pharmaceutically acceptable salt thereof, in an amount per unit form equivalent to from 0.125 mg to 20 mg, from 0.125 to 15 mg, from 0.125 mg to 12 mg, from 0.125 mg to 10 mg, from 0.125 mg to 7.5 mg, from 0.125 mg to 6 mg, from 1.6 mg to 6 mg, or from 1.625 mg to 6 mg, of pramipexole dihydrochloride monohydrate.

### The Formulations

The pharmaceutical compositions may be formulated in oral forms such as tablets or gelatin capsules wherein the statin or the 6-propylamino-4,5,6,7-tetrahydro-1,3-benzothiazole-2-amine or a pharmaceutically acceptable salt or solvate thereof or both the active ingredients are in admixture with a carrier or vehicle that may include a diluent, such as cellulose, dextrose, lactose, mannitol, sorbitol or sucrose; a lubricant, such as, acid, calcium or magnesium stearate, polyethylene glycol, silica, or talc; and if needed, a binder such as magnesium aluminum silicate, gelatin, methylcellulose, sodium carboxymethylcellulose, or polyvinylpyrrolidone.

When both of the active ingredients are present in the composition, the statin and the 6-propylamino-4,5,6,7-tetrahydro-1,3-benzothiazole-2-amine may be compounded together or separately, in the same unit form, taking care of the chemical compatibility of said active ingredients, for example by avoiding the direct contact between them according to known technologies.

For the intended use in the treatment of synucleinopathies in combination with 6-propylamino-4,5,6,7-tetrahydro-1,3-benzothiazole-2-amine, the statin is formulated in a pharmaceutical composition, wherein said statin is in admixture with a pharmaceutical carrier or vehicle.

An advantageous pharmaceutical composition according to this intended use comprises:

(a) a statin; and
(b) 6-propylamino-4,5,6,7-tetrahydro-1,3-benzothiazole-2-amine,
each in a pharmaceutical composition in admixture with a pharmaceutical carrier or vehicle; or
(a/b) a statin and 6-propylamino-4,5,6,7-tetrahydro-1,3-benzothiazole-2-amine, mixed together and in admixture with a pharmaceutical carrier or vehicle, as a fixed dose combination.

In particular, according to (a/b), the present invention provides pharmaceutical compositions including, as one of their active ingredients, an effective dose/unit form of a statin as discussed above; and, as a second active ingredient, an effective dose/unit form of 6-propylamino-4,5,6,7-tetrahydro-1,3-benzothiazole-2-amine or a pharmaceutically acceptable salt and/or solvate thereof, in admixture with a pharmaceutical carrier or vehicle.

In the pharmaceutical compositions of the present invention for oral, subcutaneous, intravenous, transdermal or topical administration, the active ingredients are preferably administered in the form of dosage units, in admixture with the classic pharmaceutical carriers or vehicles.

The dosage, i.e. the amount of active ingredient in a single dose to be administered to a patient suffering from a synucleinopathy, can vary widely depending on the age, weight, and the health condition of the patient, as also described herein above. This dosage includes the administration of a dose from 0.5 mg to 80 mg of a statin, and from 0.125 mg to 1500 mg (in pramipexole dihydrochloride monohydrate) of 6-propylamino-4,5,6,7-tetrahydro-1,3-benzothiazole-2-amine, in an IR-formulation including a (S)-enantiomer amount per unit form equivalent to from 0.125 mg to 10 mg of pramipexole dihydrochloride monohydrate, or from 0.375 mg to 3000 mg (in pramipexole dihydrochloride monohydrate) of 6-propylamino-4,5,6,7-tetrahydro-1,3-benzothiazole-2-amine, in an ER-formulation including a (S)-enantiomer amount per unit form equivalent to from 0.375 mg to 20 mg of pramipexole dihydrochloride monohydrate, according to the age of the patient, from one to three times a day by intravenous, subcutaneous, oral, or transcutaneous administration, according to the strength of the doses of the each of the active ingredients.

If the 6-propylamino-4,5,6,7-tetrahydro-1,3-benzothiazole-2-amine is pramipexole or a pharmaceutically acceptable salt thereof, said dosage is equivalent to from 0.125 mg to 20 mg or from 0.125 mg to 6 mg, normally from 1.6 mg to 6 mg, advantageously from 1.625 mg to 6 mg of pramipexole dihydrochloride monohydrate.

Generally, pharmaceutical compositions of the present invention are formulated with the classic excipients suitable for different ways of administration. Particularly advantageous are the formulations in the form of tablets, multi-score tablets, coated tables, orally disintegrating tablets, extended release tablets, hard or soft capsules, extended-release capsules, patches for transdermal administration, liquid oral solutions, syrups or suspensions in a predetermined unit form, and vials for the intravenous or subcutaneous administration.

For example, a pharmaceutical composition according to the present invention to be chronically administered in combination with 6-propylamino-4,5,6,7-tetrahydro-1,3-benzothiazole-2-amine (preferably pramipexole or a pharmaceutically acceptable salt or solvate thereof, in an amount per unit form equivalent to from 0.125 to 20 mg, normally from 0.125 mg to 6 mg, advantageously from 1.6 mg to 6 mg, preferably from 1.625 mg to 6 mg of pramipexole dihydrochloride monohydrate, and to be administered at a daily dose of from 0.375 to 20 mg, normally from 1.5 mg to 6 mg, advantageously from 1.6 mg to 6 mg, preferably from more than 4.5 mg to 6 mg, of pramipexole dihydrochloride monohydrate) comprises a statin selected from the group consisting of
atorvastatin calcium trihydrate, in an amount of from 5 mg to 80 mg, preferably from 5 mg to 60 mg;
fluvastatin sodium, in an amount of from 10 mg to 80 mg, preferably from 10 mg to 60 mg;
lovastatin, in an amount of from 10 mg to 40 mg, preferably from 10 mg to 30 mg;
pitavastatin calcium, in an amount of from 0.5 mg to 4 mg, preferably from 0.5 mg to 3 mg;
pravastatin sodium, in an amount of from 5 mg to 80 mg, preferably from 5 mg to 60 mg;
simvastatin, in an amount of from 2.5 mg to 80 mg, preferably from 2.5 mg to 60 mg; and
rosuvastatin calcium, in an amount of from 2.5 mg to 40 mg, preferably from 2.5 mg to 30 mg,
   in admixture with a pharmaceutical carrier or vehicle.

The pharmaceutical compositions may be formulated in oral forms such as tablets or gelatin capsules, wherein the statin Component (a) or the 6-propylamino-4,5,6,7-tetrahydro-1,3-benzothiazole-2-amine or pharmaceutically acceptable salt or solvate thereof Component (b); or both the active ingredients (a/b), are in admixture with a carrier or vehicle. Said carrier or vehicle may include a diluent, such as cellulose, dextrose, lactose, mannitol, sorbitol or sucrose; a lubricant, such as, acid, calcium or magnesium stearate, polyethylene glycol, silica, or talc; and if needed, a binder such as magnesium aluminum silicate, gelatin, methylcellulose, sodium carboxymethylcellulose, or polyvinylpyrrolidone, or a preservative such as methylparaben, propylparaben or butylated hydroxyanisole.

In the case of a fixed-dose combination, when the active ingredients (a/b) are each in admixture with a carrier or vehicle, it is possible to avoid the direct contact between them according to known technologies, for example as described in WO2009/154810, the contents of which are incorporated herein in their entirety by reference. For example, the fixed-dose combinations of the present invention may be formulated by mixing Component (a) with a pharmaceutical carrier or vehicle in a tablet for immediate release, and, separately, by mixing Component (b) with a pharmaceutical carrier or vehicle for extended release in another tablet. The two tablets may be introduced in a capsule for oral administration, as described for example in GB 1204580 and US 2007/0224259, the contents of both of which are incorporated herein in their entirety by reference, or in a two-piece capsule. Component (a) and Component (b), each in admixture with a pharmaceutical carrier or vehicle, may also be combined and formulated in a multi-layer tablet, as described in WO2006/089493 or in US2015/0050333, the contents of both of which are incorporated herein in their entirely by reference.

Said oral forms may be tablets coated with sucrose or with various polymers.

In the pharmaceutical combination of the present invention, common inactive ingredients of the pharmaceutical compositions include calcium carbonate, tribasic calcium phosphate, a wax, croscarmellose sodium, hydroxypropyl cellulose, lactose, lactose monohydrate, magnesium stearate, magnesium oxide, microcrystalline cellulose, hypromellose, polyethylene glycol, talc, titanium dioxide; polysorbate 80, simethicone, gelatin, pregelatinized corn starch, corn starch, mannitol, carbomer homopolymer, silicon dioxide, colloidal anhydrous silica, povidone, crospovidone, triacetin, sodium lauryl sulfate, sodium propionate, and magnesium aluminometasilicate.

Accordingly, a pharmaceutical composition Component (a) comprising a statin, preferably selected from the group consisting of the aforementioned seven statins, in the given amounts, in admixture with a pharmaceutical carrier or vehicle, may be prepared and is combined with a pharmaceutical composition Component (b) comprising a 6-propylamino-4,5,6,7-tetrahydro-1,3-benzothiazole-2-amine as follows:
(i) a member selected from the group consisting of
   pramipexole and pharmaceutically acceptable salts and solvates thereof, in a dose per unit form equivalent to from 0.125 mg to 20 mg or from 0.125 mg to 6 mg of pramipexole dihydrochloride monohydrate; and
   racemic 6-propylamino-4,5,6,7-tetrahydro-1,3-benzothiazole-2-amine and pharmaceutically acceptable salt and solvates thereof, in an amount equivalent to from 0.25 mg to 40 mg or from 0.25 mg to 12 mg of pramipexole dihydrochloride monohydrate, and
(ii) (R)-6-propylamino-4,5,6,7-tetrahydro-1,3-benzothiazole-2-amine or a pharmaceutically acceptable salt thereof, in an amount up to the total (in pramipexole dihydrochloride monohydrate) of 3000 mg.

A typical pharmaceutical combination comprises:
(a) a pharmaceutical composition comprising from 2.5 mg to 40 mg of rosuvastatin calcium in admixture with a pharmaceutical carrier in an IR-formulation; and
(b) a pharmaceutical composition comprising from 1.6 mg to 6 mg or from 1.6 mg to 2 mg of pramipexole dihydrochloride monohydrate, in admixture with a pharmaceutical carrier in an IR-formulation.

This combination is destined for the treatment of a patient suffering from a synucleinopathy by administering to said patient Component (a) once a day and Component (b) two or three times per day.

Another typical pharmaceutical combination, destined to the treatment of a patient suffering from a synucleinopathy, comprises:
(a) a pharmaceutical composition comprising from 2.5 mg to 40 mg of rosuvastatin calcium in admixture with a pharmaceutical carrier in an IR-formulation; and
(b) a pharmaceutical composition comprising from 1.5 mg to 13.5 mg or from 1.5 mg to 4.5 mg of pramipexole dihydrochloride monohydrate, in admixture with a pharmaceutical carrier in an ER-formulation.

Component (a) and Component (b) of this combination will be administered, concurrently or sequentially, once a day.

A particularly efficacious pharmaceutical combination, destined to be administered to a patient suffering from a synucleinopathy, comprises:
(a) a pharmaceutical composition comprising from 2.5 mg to 40 mg of rosuvastatin calcium in admixture with a pharmaceutical carrier or vehicle in an IR-formulation; and
(b) a pharmaceutical composition comprising pramipexole or a pharmaceutically acceptable salt or solvate thereof, in an amount equivalent to from more than 4.5 mg to 20 mg or from more than 4.5 mg to 6 mg of pramipexole dihydrochloride monohydrate, in admixture with a pharmaceutical carrier in an ER-formulation.

In this combination, each of the Components (a) and (b) is formulated with a pharmaceutical carrier for a once a day, concurrent or sequential administration. Component (b) may be formulated with a pharmaceutical carrier or vehicle delivering pramipexole once a day orally or transdermally, for example in a transdermal therapeutic system delivering pramipexole in an amount equivalent to from more than 4.5 mg to 20 mg or from more than 4.5 mg to 6 mg of pramipexole dihydrochloride dihydrate.

Another typical pharmaceutical combination comprises:
(a) a pharmaceutical composition comprising from 2.5 mg to 40 mg of rosuvastatin calcium in admixture with a pharmaceutical carrier in an IR-formulation; and
(b) a pharmaceutical composition comprising a mixture of from 1.6 to 10 mg or from 1.6 to 2 mg of pramipexole dihydrochloride monohydrate, and of an amount of (R)-6-propylamino-4,5,6,7-tetrahydro-1,3-benzothiazole-2-amine dihydrochloride monohydrate to reach a total (in pramipexole dihydrochloride monohydrate) of 300 mg, in admixture with a pharmaceutical carrier in an IR-formulation.

This combination is destined to the treatment of a patient suffering from a synucleinopathy by administering to said patient Component (a) once a day and Component (b) two or three times per day. The (R)-6-propylamino-4,5,6,7-tetrahydro-1,3-benzothiazole-2-amine dihydrochloride monohydrate active ingredient may be prepared according to US 2012/0253047, the contents of which are incorporated herein in their entirety by reference.

The combination of a statin with 6-propylamino-4,5,6,7-tetrahydro-1,3-benzothiazole-2-amine includes fixed-dose combinations wherein said statin and said 6-propylamino-4,5,6,7-tetrahydro-1,3-benzothiazole-2-amine are combined in the same unit form.

Accordingly, in said unit form, Component (a) comprising a statin, preferably selected from the group consisting of atorvastatin calcium trihydrate, in an amount of from 5 mg to 80 mg, preferably from 5 mg to 60 mg; fluvastatin sodium, in an amount of from 10 mg to 80 mg, preferably from 10 mg to 60 mg; lovastatin, in an amount of from 10 mg to 40 mg, preferably from 10 mg to 30 mg; pitavastatin calcium, in an amount of from 0.5 mg to 4 mg, preferably from 0.5 mg to 3 mg; pravastatin sodium, in an amount of from 5 mg to 80 mg, preferably from 5 mg to 60 mg; simvastatin, in an amount of from 2.5 mg to 80 mg, preferably from 2.5 mg to 60 mg; and rosuvastatin calcium, in an amount of from 2.5 mg to 40 mg, preferably from 2.5 mg to 30 mg, in admixture with a pharmaceutical carrier or vehicle for immediate release, is combined with Component (b), comprising 6-propylamino-4,5,6,7-tetrahydro-1,3-benzothiazole-2-amine, in an amount equivalent to from 0.375 mg to 3000 mg of pramipexole dihydrochloride monohydrate, including a S-enantiomer amount per unit form equivalent to from 0.375 mg to 20 mg, normally from 0.375 mg to 6 mg, of pramipexole dihydrochloride monohydrate, in admixture with a pharmaceutical carrier or vehicle for extended release, in the same dosage unit form.

Advantageously, in this fixed-dose combination, said statin Component (a), in said amount in an IR-formulation, is combined with Component (b), comprising 6-propylamino-4,5,6,7-tetrahydro-1,3-benzothiazole-2-amine, in an amount equivalent to from 1.5 mg to 3000 mg of pramipexole dihydrochloride monohydrate, in an ER-formulation, including a S-enantiomer amount equivalent to from 1.5 mg to 20 mg, normally from 1.5 mg to 6 mg, of pramipexole dihydrochloride monohydrate.

Preferably, in this fixed-dose combination, said statin Component (a), in said amount in an IR-formulation, is combined with Component (b), comprising a 6-propylamino-4,5,6,7-tetrahydro-1,3-benzothiazole-2-amine selected from the group consisting of pramipexole and pharmaceutically acceptable salts and solvates thereof, in an amount equivalent to from 1.5 mg to 20 mg, normally from 1.5 mg to 6 mg, of pramipexole dihydrochloride monohydrate, in an ER-formulation.

This fixed-dose combination is destined to the treatment of a patient suffering from a synucleinopathy by administering it to said patient once a day.

A typical pharmaceutical fixed-dose combination comprises:
(a) a pharmaceutical composition comprising from 2.5 mg to 40 mg of rosuvastatin calcium in admixture with a pharmaceutical carrier in an IR-formulation; and
(b) a pharmaceutical composition comprising from 1.5 to 20 mg, normally from 1.5 to 6 mg of pramipexole dihydrochloride monohydrate, in admixture with a pharmaceutical carrier in an ER-formulation,
   in a dosage unit form, such as a bi-layer tablet, for the simultaneous administration of Component (a) and Component (b), concurrently delivering said statin, in immediate release and said pramipexole, in sustained release.

### Kits

The present invention also provides a kit or package containing a medicament, a pharmaceutical combination, or a pharmaceutical composition as described herein, accompanied by instructions for use of the same in the treatment of a synucleinopathy in a patient in need thereof.

In one embodiment, a kit of the present invention is a kit comprising a combination of a statin and 6-propylamino-4,5,6,7-tetrahydro-1,3-benzothiazole-2-amine formulated together in a pharmaceutical composition in admixture with a pharmaceutical carrier or vehicle; and instructions for use of the same for treatment of a synucleinopathy in a patient in need thereof.

In another embodiment, a kit of the present invention is a kit comprising pharmaceutical composition (a) comprising a statin and pharmaceutical composition (b) comprising 6-propylamino-4,5,6,7-tetrahydro-1,3-benzothiazole-2-amine; and instructions for use of the same for treatment of a synucleinopathy in a patient in need thereof.

### EXAMPLES

### Example 1

A Phase I-II clinical study was conducted in parkinsonian subjects receiving oral doses of pramipexole or rosuvastatin, alone and in combination.

The objective of the study was to demonstrate that pramipexole and rosuvastatin, when administered together at their standard therapeutic doses, can safely normalize concentrations of synuclein species in peripheral blood exosomes.

To be enrolled in the study, male or female participants (40 to 89 years of age) were required to carry the diagnosis of Parkinson's disease or a related synucleinopathic disorder. All subjects signed an informed consent form indicating that they understood the purpose of and procedures required for the study and that they were willing to participate in the study and comply with all study procedures and restrictions. Key criteria for exclusion of a subject from enrollment in the study were as follows:
1. Any clinically relevant acute or chronic disease which could interfere with the subjects' safety during the trial, expose them to undue risk, or interfere with the study objectives.
2. History or presence of gastrointestinal, hepatic, or renal disease or other condition known to interfere with the absorption, distribution, metabolism or excretion of the study medications;
3. History of substance abuse, known drug addiction, or positive test for drugs of abuse or alcohol.
4. History of drug or other significant allergy.
5. Excessive daily consumption of xanthines containing drinks (i.e. >500 mg/day of caffeine).
6. Hospitalization or intake of an investigational drug within 30 days of study entry.

Following baseline clinical and laboratory evaluations, consenting individuals meeting accession criteria were first randomized to treatment with pramipexole titrated up to maximum tolerated dose (MTD), or up to a maximum dose of 5 mg/day, whichever came first. Patients were then maintained on pramipexole at their MTD or at 5 mg/day for 2 to 4 weeks. At the end of the maintenance period, venous blood for synuclein and drug assays was collected and patients were randomized to either rosuvastatin treatment (starting with 20 mg/day for approximately 2 weeks. If 20 mg/day was tolerated the dose of rosuvastatin was then to be increased to 40 mg/day (maximum recommended dose)) or placebo added on to pramipexole treatment. Patients were stably maintained on pramipexole and rosuvastatin (or placebo) treatment for 6 to 12 weeks. At the end of this combination treatment period venous blood for synuclein and drug assays was collected. Doses of both drugs were then tapered in accordance with current recommendations and patients were returned to their pre-admission regimen pending discharge from the study.

Drug safety-tolerability was monitored throughout the trial by means of standard clinical and laboratory tests. Weekly telephone interviews were generally conducted on those not scheduled for a clinic visit. A final safety check was performed approximately one month after withdrawal of all study medications.

Additionally, venous blood for synuclein and drug assays were collected during the study.

Results surprisingly showed that the oral administration of a combination of pramipexole and rosuvastatin was associated with a tendency to normalize the characteristic alterations in synuclein and synuclein congener concentrations in exosomes collected from peripheral venous blood samples from patients who safely tolerated their therapeutic regimens.

In conclusion, the co-administration of standard approved doses of pramipexole and rosuvastatin yielded clear evidence of a drug-combination-induced tendency to normalize synuclein processing indicative of a reduction in toxic species formation in the central nervous system of a type associated with a neuroprotective efficacy that clinically benefits patients suffering from Parkinson's disease or a related synucleinopathy.

### REFERENCES

Bar-On et al. 2008: Bar-On P, Crews L, Koob A O, Mizuno H, Adame A, Spencer B, Masliah E; “Statins reduce neuronal alpha-synuclein aggregation in in vitro models of Parkinson's disease”; J Neurochem. 2008 June; 105(5):1656-67.
Butterfied D A et al. 2011: Butterfied D A, Barone E, Mancuso C; “Cholesterol-Independent Neuroprotective And Neurotoxic Activities Of Statins: Perspectives For Statin Use In Alzheimer Disease And Other Age-Related Neurodegenerative Disorders”; Pharmacol Res 2011 September; 64(3): 180-186.
Corrigan et al. 2000: Corrigan M H, Denahan A Q, Wright C E, Ragual R J, Evans D L; Corrigan M H, Denahan A Q, Wright C E, Ragual R J, Evans D; “Comparison of pramipexole, fluoxetine, and placebo in patients with major depression”; Depress Anxiety. 2000; 11(2):58-65.
Kang S Y et al. 2017: Kang S Y, Lee S B, Kim H J, Kim H T, Yang H O, Jang W; “Autophagic modulation by rosuvastatin prevents rotenone-induced neurotoxicity in an in vitro model of Parkinson's disease”; Neurosci Lett. 2017 Mar. 6; 642:20-26).
Kim et al. 2004: Kim S, Seo J H, Suh Y H, “Alpha-synuclein, Parkinson's disease, and Alzheimer's disease”; Parkinsonism Relat. Disord. 2004 May; 10 Suppl. 1: S9-13.
Koob A O et al. 2010: Koob A O, Ubhi K, Paulsson J F, Kelly J, Rockenstein E, Mante M, Adame A, Masliah E; “Lovastatin ameliorates alpha-synuclein accumulation and oxidation in transgenic mouse models of alpha-synucleinopathies”; Exp Neurol. 2010 February; 221(2):267-74.
Luo et al. 2016: Luo H T, Zhang J P, Miao F; “Effects of pramipexole treatment on the α-synuclein content in serum exosomes of Parkinson's disease patients”; Exp Ther Med. 2016 September; 12(3):1373-1376.
Marques and Outeiro 2012: Marques O, Outeiro T F; “Alpha-synuclein: from secretion to dysfunction and death”; Cell Death Dis. 2012 Jul. 19; 3:e350. doi: 10.1038/cddis.2012.94.
Orr J D 2008: Orr J; “Statins in the spectrum of neurologic disease” Curr Atheroscler Rep. 2008 February; 10(1):11-8.
Saedi Saravi S S et al. 2017: Saeedi Saravi S S, SaeediSaravi S S, Khoshbin K, Dehpour A R; “Current insights into pathogenesis of Parkinson's disease: Approach to mevalonate pathway and protective role of statins”; Biomed Pharmacother. 2017 Apr. 15; 90:724-730.
Schneider C S and Mierau J, 1987: Schneider C S, Mierau J “Dopamine autoreceptor agonists: resolution and pharmacological activity of 2,6-diaminotetrahydrobenzothiazole and an aminothiazole analogue of apomorphine”. J. Med Chem. 1987 March; 30(3):494-8.
Shapira et al. 2013: Schapira A H, McDermott M P, Barone P, Comella C L, Albrecht S, Hsu H H, Massey D H, Mizuno Y, Poewe W, Rascol O, Marek K. “Pramipexole in patients with early Parkinson's disease (PROUD): a randomised delayed-start trial”; Lancet Neurol. 2013 August; 12(8):747-55).
Shi et al. 2014: Shi M, Liu C, Cook T J, Bullock K M, Zhao Y, Ginghina C, Li Y, Aro P, Dator R, He C, Hipp M J, Zabetian C P, Peskind E R, Hu S C, Quinn J F, Galasko D R, Banks W A, Zhang J; “Plasma exosomal α-synuclein is likely CNS-derived and increased in Parkinson's disease”; Acta Neuropathol. 2014 November; 128(5):639-50. doi: 10.1007/s00401-014-1314-y. Epub 2014 Jul. 6.

1. A method for the treatment of a synucleinopathy in a patient in need of said treatment, which comprises administering to said patient a statin, in combination with 6-propylamino-4,5,6,7-tetrahydro-1,3-benzothiazole-2-amine or a pharmaceutically acceptable salt or solvate thereof.

2. The method of claim 1, wherein said statin is administered to said patient at a daily dose of from 0.5 mg to 80 mg and said 6-propylamino-4,5,6,7-tetrahydro-1,3-benzothiazole-2-amine or pharmaceutically acceptable salt or solvate thereof is administered to said patient at a daily dose equivalent to from 0.375 mg to 3000 mg of pramipexole dihydrochloride monohydrate, including a (S)-enantiomer daily dose equivalent to from 0.375 mg to 20 mg of pramipexole dihydrochloride monohydrate.

3. The method of claim 1, wherein
said statin is selected from the group consisting of atorvastatine and pharmaceutically acceptable salts and solvates thereof, administered at a daily dose equivalent to from 5 mg to 80 mg of atorvastatin free acid; fluvastatin and pharmaceutically acceptable salts and solvates thereof, administered at a daily dose equivalent to from 10 mg to 80 mg of fluvastatin free acid; lovastatin, administered at a daily dose of from 5 mg to 80 mg; pitavastatin and pharmaceutically acceptable salts and solvates thereof, administered at a daily dose equivalent to from 0.5 mg to 4 mg of pitavastatin free acid; pravastatin and pharmaceutically acceptable salts and solvates thereof, administered at a daily dose of from 2.5 mg to 60 mg; simvastatin, administered at a daily dose of from 2.5 mg to 40 mg; and rosuvastatin and pharmaceutically acceptable salts and solvates thereof, administered at a daily dose of from 2.5 mg to 40 mg; and
said 6-propylamino-4,5,6,7-tetrahydro-1,3-benzothiazole-2-amine is selected from the group consisting of pramipexole and pharmaceutically acceptable salts and solvates thereof, at a daily dose equivalent to a range selected from the group consisting of from 0.375 mg to 20 mg, from 0.375 to 15 mg, from 0.375 mg to 12 mg, from 0.375 mg to 10 mg, from 0.375 mg to 7.5 mg, from 0.375 mg to 6 mg, from 1.6 mg to 6 mg, from 1.625 mg to 6 mg, or from 3 mg to 6 mg of pramipexole dihydrochloride monohydrate.

4. The method of claim 1, wherein
said statin is administered to said patient in a pharmaceutical composition in dosage unit form comprising said statin, selected from the group consisting of atorvastatine and pharmaceutically acceptable salts and solvates thereof, in an amount per unit form equivalent to from 5 mg to 80 mg of atorvastatin free acid; fluvastatin and pharmaceutically acceptable salts and solvates thereof, in an amount per unit form equivalent to from 10 mg to 80 mg of fluvastatin free acid; lovastatin, in an amount per unit of from 5 mg to 80 mg; pitavastatin and pharmaceutically acceptable salts and solvates thereof, in an amount per unit form equivalent to from 0.5 mg to 4 mg of pitavastatin free acid; pravastatin and pharmaceutically acceptable salts and solvates thereof, in an amount per unit form equivalent to from 2.5 mg to 60 mg of pravastatin sodium; simvastatin, in an amount per unit form of from 2.5 mg to 40 mg; and rosuvastatin and pharmaceutically acceptable salts and solvates thereof, in an amount per unit form equivalent to from 2.5 mg to 40 mg of rosuvastatin calcium,
in admixture with a pharmaceutical carrier or vehicle; and
said 6-propylamino-4,5,6,7-tetrahydro-1,3-benzothiazole-2-amine is administered to said patient in a pharmaceutical composition in dosage unit form comprising said 6-propylamino-4,5,6,7-tetrahydro-1,3-benzothiazole-2-amine, selected from the group consisting of pramipexole and pharmaceutically acceptable salts and solvates thereof, in an amount per unit form equivalent to a range selected from the group consisting of from 0.125 mg to 20 mg, from 0.125 to 15 mg, from 0.125 mg to 12 mg, from 0.125 mg to 10 mg, from 0.125 mg to 7.5 mg, from 0.125 mg to 6 mg, from 1.6 mg to 6 mg, from 1.625 mg to 6 mg, and from 3 mg to 6 mg of pramipexole dihydrochloride monohydrate,
in admixture with a pharmaceutical carrier or vehicle.

5. The method of claim 4, wherein said statin is rosuvastatin calcium, in an amount per unit form of from 2.5 mg to 40 mg.

6. The method of claim 1, wherein said statin and said 6-propylamino-4,5,6,7-tetrahydro-1,3-benzothiazole-2-amine are concurrently administered to said patient in a fixed-dose combination, in a single dosage unit form, wherein said statin and said 6-propylamino-4,5,6,7-tetrahydro-1,3-benzothiazole-2-amine or a pharmaceutically acceptable salt or solvate thereof are mixed in said single unit form and in admixture with a pharmaceutical carrier or vehicle.

7. The method of claim 6, wherein, in said unit form,
said statin is selected from the group consisting of atorvastatin calcium trihydrate, in an amount of from 5 mg to 80 mg, fluvastatin sodium, in an amount of from 10 mg to 80 mg; lovastatin, in an amount of from 10 mg to 40 mg; pitavastatin calcium, in an amount of from 0.5 mg to 4 mg; pravastatin sodium, in an amount of from 5 mg to 80 mg; simvastatin, in an amount of from 2.5 mg to 80 mg; and rosuvastatin calcium, in an amount of from 2.5 mg to 40 mg, in admixture with a pharmaceutical carrier or vehicle for immediate release; and
said 6-propylamino-4,5,6,7-tetrahydro-1,3-benzothiazole-2-amine is selected from the group consisting of pramipexole and pharmaceutically acceptable salts and solvates thereof, in an amount equivalent to from 1.5 mg to 20 mg of pramipexole dihydrochloride monohydrate, in admixture with a pharmaceutical carrier or vehicle for extended release.

8. The method of claim 7, wherein, in said unit form, said pramipexole or pharmaceutically acceptable salts and solvates thereof, is pramipexole dihydrochloride monohydrate, in an amount of from 1.5 mg to 6 mg.

9. The method of claim 7, wherein, in said unit form, said statin is rosuvastatin calcium, in an amount of from 2.5 mg to 40 mg and said pramipexole or pharmaceutically acceptable salts and solvates thereof, is pramipexole dihydrochloride monohydrate, in an amount of from 1.5 mg to 20 mg.

10. The method of claim 9, wherein, in said unit form, said pramipexole dihydrochloride monohydrate is in an amount of from 1.5 mg to 6 mg.

11. The method of claim 1, wherein said synucleinopathy is selected from the group consisting of Parkinson's disease, Lewy body dementia, dementia with Lewy bodies, Alzheimer's disease, the Lewy body variant of AD, multiple system atrophy, neurodegeneration with brain iron accumulation, and parkinsonian disorders associated with glucocerebrosidase (GBA) mutations.

12. A pharmaceutical combination comprising a statin and 6-propylamino-4,5,6,7-tetrahydro-1,3-benzothiazole-2-amine or a pharmaceutically acceptable salt and/or solvate thereof.

13. A pharmaceutical composition comprising a statin and 6-propylamino-4,5,6,7-tetrahydro-1,3-benzothiazole-2-amine or a pharmaceutically acceptable salt and/or solvate thereof.

14. The composition of claim 14, further comprising a pharmaceutically acceptable carrier or vehicle.

15. A kit comprising the pharmaceutical combination of claim 12, or the pharmaceutical composition of claim 13, and instructions for treatment of a synucleinopathy in a patient in need thereof.

16. The pharmaceutical combination of claim 12, wherein said statin is present at a daily dose of from 0.5 mg to 80 mg and said 6-propylamino-4,5,6,7-tetrahydro-1,3-benzothiazole-2-amine or pharmaceutically acceptable salt or solvate thereof is present at a daily dose equivalent of from 0.375 mg to 3000 mg of pramipexole dihydrochloride monohydrate.

17. The pharmaceutical combination of claim 12, wherein said statin is present at a daily dose of from 0.5 mg to 80 mg and said 6-propylamino-4,5,6,7-tetrahydro-1,3-benzothiazole-2-amine or pharmaceutically acceptable salt or solvate thereof is a (S)-enantiomer present at a daily dose equivalent to from 0.375 mg to 20 mg of pramipexole dihydrochloride monohydrate.

18. The pharmaceutical composition of claim 13, wherein said statin is present at a daily dose of from 0.5 mg to 80 mg and said 6-propylamino-4,5,6,7-tetrahydro-1,3-benzothiazole-2-amine or pharmaceutically acceptable salt or solvate thereof is present at a daily dose equivalent of from 0.375 mg to 3000 mg of pramipexole dihydrochloride monohydrate.

19. The pharmaceutical composition of claim 13, wherein said statin is present at a daily dose of from 0.5 mg to 80 mg and said 6-propylamino-4,5,6,7-tetrahydro-1,3-benzothiazole-2-amine or pharmaceutically acceptable salt or solvate thereof is a (S)-enantiomer present at a daily dose equivalent to from 0.375 mg to 20 mg of pramipexole dihydrochloride monohydrate.
